(19) United States
(12) Patent Application Publication
(10) Pub. No.: US 2018/0120329 A1
(43) Pub. Date: May 3, 2018

(54) CXCL13 AS AN INDICATOR OF GERMINAL ACTIVITY AND IMMUNE RESPONSE

(71) Applicant: La Jolla Institute for Allergy and Immunology, La Jolla, CA (US)

(72) Inventor: SHANE CROTTY, San Diego, CA (US)

(73) Assignee: La Jolla Institute for Allergy and Immunology, La Jolla, CA (US)

(21) Appl. No.: 15/561,920

(22) PCT Filed: Mar. 25, 2016
(86) PCT No.: PCT/US2016/024255

§ 371 Date: Sep. 26, 2017

Related U.S. Application Data
(60) Provisional application No. 62/138,230, filed on Mar. 25, 2015.

(51) Int. Cl. G01N 33/68

(52) U.S. Cl. CPC G01N 33/6863; G01N 2333/522; G01N 2800/104; G01N 2800/102; G01N 2800/24

(57) ABSTRACT

Presented herein are methods of detecting and/or monitoring germinal center activity in a subject according to an amount of CXCL13 in the blood of a subject. Also presented herein are methods of determine the efficacy of a vaccine or antigen at inducing an immune response. Methods are also presented for monitoring, screening, and/or diagnosing an autoimmune disorder or immune-suppression in a subject and for monitoring or adjusting a treatment.

### RELATED PATENT APPLICATION(S)

This patent application claims the benefit of U.S. Provisional Patent Application No. 62/138,230 filed on Mar. 25, 2015, entitled CXCL13 AS A BIOMARKER OF GERMINAL CENTER ACTIVITY FOR HUMAN VACCINE EVALUATION, naming Shane Crotty as inventor, and designated by Attorney Docket No. 051501-0437936.

This invention was made with government support by grant numbers U19AI090970 and UM1AI100663 awarded by the National Institutes of Health (NIH). The government has certain rights in the invention.

### FIELD

Embodiments of the invention relate to certain methods of detecting the efficacy of an antigen or vaccine treatment, and/or detecting germinal center activity and/or detecting T helper cell activity in a patient in response to treatment. In some embodiments a method herein comprises determining an amount, or a change in the amount of, CXCL13 (chemokine (C—X—C motif) ligand 13) in a patient. Certain embodiments relate to a method of detecting increased germinal center activity or Tfh activity, and diagnosing or treating an autoimmune disorder.

### SUMMARY

Presented herein, in some aspects, is a method of detecting an immune response in a subject to an antigen or vaccine, the method comprising, a) determining a first amount of CXCL13 in a first sample obtained from a subject; b) administering an antigen or vaccine to the subject; c) determining a second amount of CXCL13 in a second sample obtained from the subject after the administering in (b); and d) comparing the first and the second amount of CXCL13, wherein a presence or absence of an immune response to the antigen or vaccine is determined according to the comparison. In certain embodiments, the comparison comprises determining the presence or absence of an increase in the second amount of CXCL13 compared to the first amount of CXCL13. In some embodiments, the presence of an increase in the second amount of CXCL13 indicates the presence of an immune response to the antigen or vaccine. In some embodiments, the presence of the immune response to the antigen or vaccine comprises an increase in germinal center (GC) activity. In certain embodiments, an increase in germinal center (GC) activity comprises at least a 10% increase in the amount of germinal centers in one or more lymph node tissues in the subject. In certain embodiments, an increase in germinal center (GC) activity comprises at least a 10% increase in the amount of Tfh cells in one or more lymph node tissue in the subject. In some embodiments, the Tfh cells produce CXCL13. In certain embodiments, an increase in germinal center (GC) activity comprises at least a 10% increase in the amount of circulating activated T-cells in the subject, wherein the activated T-cells are ICOS+PD1+CXCR5+CD4+. In certain embodiments, the comparing in (d) comprises determining a ratio of the first amount of CXCL13 to the second amount of CXCL13. In certain embodiments, a second sample is obtained at least 1 week after administering the antigen or vaccine. In certain embodiments, the first and the second sample comprise or consist essentially of blood, plasma or serum obtained from the patient. In certain embodiments, the determining of (b) and (c) comprises use of a binding agent that specifically binds to CXCL13. In certain embodiments, the binding agent comprises an antibody or a fragment of an antibody that binds to specifically to CXCL13. In certain embodiments, the antigen or vaccine comprises a pathogen, or an immunogenic portion thereof. In certain embodiments, the pathogen comprises a live, dead or attenuated virus, bacteria, fungus or parasite. In certain embodiments, the immunogenic portion of the pathogen comprises a viral, bacterial, fungal or parasite extract or protein. In certain embodiments, the presence of an immune response indicates the presence of circulating antibodies in the subject that bind specifically to the antigen, or to an antigen component of the vaccine. In certain embodiments, the amount of circulating antibodies in the subject after the administering of (b) is substantially larger than an amount of circulating antibodies in the subject prior to the administering of (b). In certain embodiments, the presence of an increase in the second amount of CXCL13 compared to the first amount of CXCL13 is at least a 10% increase in the second amount of CXCL13 compared to the first amount of CXCL13. In certain embodiments, the absence of an increase in the second amount of CXCL13 indicates the absence of an immune response to the antigen or vaccine. In certain embodiments, the absence of an immune response indicates the subject is not responsive to the antigen or vaccine. In certain embodiments, the absence of an immune response indicates the subject is immuno-suppressed or immuno-incompetent. In certain embodiments, the presence of an immune response indicates the vaccine is effective.

In some aspects, presented herein is a method of treating a subject with an autoimmune disorder comprising, a) providing a subject having an autoimmune disorder wherein the disorder is characterized by the presence of autoantibodies; b) determining a first amount of CXCL13 in a sample obtained from the subject; c) administering a dose of a drug to the subject, wherein the drug is configured to treat the autoimmune disorder; d) determining a second amount of CXCL13 in a second sample obtained from the subject after the administering of (c); and e) comparing the first amount to the second amount thereby providing a comparison. In certain embodiments, if the second amount of CXCL13 is less than the first amount of CXCL13, continue administering the drug. In certain embodiments, if the second amount of CXCL13 is the same or larger than the first amount of CXCL13, discontinue administering the drug, or increase the dose of the drug administered.

In some aspects, presented herein is a method of diagnosing a subject with an autoimmune disorder comprising, a) providing a first sample comprising a known amount of CXCL13; b) determining an amount of CXCL13 in a second sample obtained from a subject having or suspected of having an autoimmune disorder; c) comparing the amount of CXCL13 in the first sample to the second sample, thereby providing a comparison; and d) determining the presence or absence of an autoimmune disorder in the subject according to the comparison. In some embodiments, the presence of an autoimmune disorder is determined and the amount of CXCL13 in the second sample is at least 30% greater than the amount of CXCL13 in the first sample. In some embodiments the absence of an autoimmune disorder is determined and the amount of CXCL13 in the second sample is not significantly different than the amount of CXCL13 in the first sample. In some embodiments the autoimmune disorder is characterized by the presence of autoantibodies. In some embodiments the autoimmune disorder is systemic lupus erythematosus or rheumatoid arthritis. In certain embodiments a sample comprises or consists essentially of blood, plasma or serum obtained from the subject, and wherein the subject is a human subject.

### BRIEF DESCRIPTION OF THE DRAWINGS

FIG. 1A shows detection of germinal center (GC) Tfh cells in non-draining lymph nodes (LN)(FIG. 1A, left panel) and draining LN (FIG. 1A, right panel) as PD-1hi/CXCR5hi cells (gated on CD4+CD3+) in macaque after immunization with TLR nanoparticles. FIG. 1B shows plasma amounts of CXCL13 (pg/ml)(y-axis) correlated with percent GC Tfh cells (x-axis) in LN in multiple subjects after immunization with TLR nanoparticles.

FIG. 2 shows CXCL13 is the ligand for CXCR5 and expressed by GC Tfh cells.

FIG. 3 shows CXCL13 is elevated in the plasma of Protocol C top neutralizers.

FIG. 4 shows serum CXCL13 correlates with lymph node germinal centers in mice.

FIG. 5 shows serum CXCL13 after immunization in rhesus macaque.

FIG. 6 shows increased GC Tfh cells after immunization.

FIG. 7 shows plasma CXCL13 is increased 1 week after immunization.

FIG. 8 shows CXCL13 correlates with GC Tfh cells in macaques.

FIG. 9 shows increased CXCL13 after immunization is adjuvant dependent.

FIG. 10 shows increases in CXCL13 after immunization is correlated with Ab binding titers and bone marrow plasma cells.

FIG. 11 shows BG505 SOSIP macaque trial, as shown below.

FIG. 12 demonstrates that CXCL13 is higher in top neutralizers than low neutralizers. CXCL13 correlates with germinal center activity in mice and macaques; and bone marrow plasma cells and antibody levels in macaques.

FIG. 13—Plasma CXCL13 in HIV+ neutralizers and non-neutralizers.

FIG. 13A shows HIV neutralizers grouped by neutralization score for IAVI protocol C. The neutralization score is derived from both breadth and potency data from a 37 virus cross-clade pseudo-virus panel. 228 individuals were tested for neutralizing antibodies at time points greater than 4 years point infection. At this time point, most individuals have developed maximum breadth. FIG. 13B shows plasma CXCL13 from top (neutralization score >1.0) and low (neutralization score <0.5) neutralizing individuals at the earliest time point available after infection (range 1-9 months; mean 4 months). FIG. 13C shows plasma CXCL13 from top and low neutralizing individuals at the time of bnAb generation (range 24-54 months; mean 40 months). Limit of detection (LOD) was 8 pg/ml for these assays. Mean and intraquartile ranges are shown in FIGS. 13B and 13C. Each point represents an individual.

FIG. 14—Correlation of plasma CXCL13 and lymphoid GC Tfh in humans.

FIG. 14A shows identification of GC Tfh cells as PD-1hiCXCR5hi (gated on CD4+CD3+) T cells in human tonsil (FIG. 14A, left panel) and intracellular cytokine staining for CXCL13 in unstimulated GC Tfh and all other non-GC Tfh CD4+ T cells in human tonsil (FIG. 14A, right panels). FIG. 14B shows intracellular cytokine staining for CXCL13 in unstimulated cell subsets from 9 human tonsils. The x-axis indicates GC Tfh cells, all other non-GC Tfh CD4+ T cells, CD19+ B cells, all other non-CD4+ and non-CD19+ cells. Note: Macrophages, dendritic cells, and follicular dendritic cells may be underrepresented or missing in these tissue preparations. FIG. 14C shows intracellular cytokine staining for CXCL13 in unstimulated GC Tfh cells in human spleen (representative of 2 analyzed) and a human lymph node. FIG. 14D identification of GC Tfh cells as PD-1hiCXCR5hi (gated on CD4+CD3+) T cells in human inguinal lymph node. FIG. 14E shows matched plasma CXCL13 and lymph node GC Tfh cells in 14 human donors. ART=HIV positive anti-retroviral treated; HIV neg=HIV seronegative; VC=HIV positive viremic controller.

FIG. 15—Plasma CXCL13 levels in mice after immunization.

FIG. 15A shows plasma CXCL13 in naïve mice or 7 days after Alum+NP-OVA footpad immunization, LCMV infection, or vaccinia virus infection. Closed circles indicate the presence of the transferred CD4+ TCR transgenic cells into recipient hosts before transfer. OT-II cells were transferred into the Alum+NP-OVA group and NIP cells were transferred into the LCMV group. Open circles indicate untransferred mice. FIG. 15B shows identification of GC Tfh cells as PD-1hiCXCR5hi (gated on CD4+) cells in the draining popliteal lymph node of an NP-OVA immunized mouse. FIG. 15C shows correlation of plasma CXCL13 and GC Tfh cells in naïve and Alum+OVA immunized mice in panel A. GC Tfh cells in naïve mice were set at the limit of detection (0.1% of total CD4+ T cells).

FIG. 15 D-E—Plasma CXCL13 in B6 mice immunized in the footpad with KLH+Alum. Data representative of 2 experiments of 10 mice each. (D) Plasma CXCL13 pre- and post primary immunization. FIG. 15E shows plasma CXCL13 in the same mice as in FIG. 15D after re-immunization with KLH+Alum at 50 days post primary immunization. FIG. 15F shows correlation of plasma CXCL13 and GC Tfh in the draining popliteal lymph node at 10 and 18 days post re-immunization, from animals in FIG. 15E. CXCL13 is plotted as fold change of day 10 or 18 post booster immunization over pre-boost (d50) CXCL13.

FIG. 16—GC Tfh cells in rhesus macaques correlate with plasma CXCL13.

FIG. 16A shows identification and characterization of GC Tfh cells (PD1hiCXCR5hi CD4+ T cells) in lymph node from rhesus macaques. Expression of Bcl-6, ICOS, and CD200 is shown for GC Tfh (red), CXCR5+ Tfh cells (blue), and non-Tfh cells (filled gray). FIG. 16B shows identification of GC Tfh cells (left panels) and GC B cells (right panels, identified as Ki67+Bcl6+ cells, gated on CD20+ cells) in non-draining (axillary) and draining inguinal lymph nodes (popliteal) of macaques 7 days after protein+adjuvant immunization in the leg. FIG. 16C shows correlation of plasma CXCL13 and GC Tfh cells in the draining inguinal lymph node of macaques 7 days after the 2nd or 3rd protein+adjuvant immunization. Alum or TLR-encapsulated PLGA nanoparticles were used as adjuvants. All data are representative to 2 similar immunization experiments in rhesus macaques totaling 22 animals.

FIG. 17—Plasma CXCL13 is increased after immunization in humans.

FIG. 17A shows plasma CXCL13 measured before immunization (day 0) and 7 days post yellow fever vaccination in 17 individuals. FIG. 17 (B-E)—HVTN 068 participants who received an Ad5-vector encoding HIV gag and envelope immunization 6 months post prime. FIG. 17B shows kinetic analysis of plasma CXCL13 post Ad5/HIV boost. Plasma CXCL13 was measured in 11 vaccinated individuals. FIG. 17C shows correlation of plasma CXCL13 seven days post immunization and anti-gp140 (ConS; consensus group M) Env antibody responses (ELISA OD) four weeks post immunization in 18 vaccinated individuals. Anti-gp41 antibody ELISA OD is background subtracted. FIG. 17D shows correlation of plasma CXCL13 seven days post immunization and anti-gp41 Env antibody responses (ELISA OD) four weeks post immunization in 25 vaccinated individuals. Anti-gp41 antibody ELISA OD is background subtracted. FIG. 17E shows correlation of plasma CXCL13 seven days post immunization and the fold change of ICOS+ blood Tfh-like cells (% at day 7 post boost over % at pre-boost time point; Heit A, McElrath M J, submitted) in 6 vaccinated donors.

FIG. 18—Analysis of HIV viral load and plasma CXCL13 as independent variables correlated with generation of bnAb against HIV. FIG. 18A shows HIV viral load (copies/ml) of the HIV+ individuals in FIG. 13B with an HIV neutralization score of >1 or <0.5 at the ˜4 month time point. Mean is indicated by a bar. FIG. 18B shows HIV viral load (copies/ml) of the individuals in FIG. 13C with an HIV neutralization score of >1 or <0.5 at the ˜40 month time point. Mean is indicated by a bar. FIG. 18C shows ANCOVA results showing adjusted means (adj mean) and P values for ˜4 month (Early) and ˜40 month (Late) samples sets.

FIG. 19—Plasma CXCL13 is not significantly increased after influenza immunization in humans.

FIG. 19A shows plasma CXCL13 measured before immunization (day 0) and 7 days post influenza vaccination in 10 individuals. Healthy donors were enrolled in an influenza vaccine study at the Stanford-Lucile Packard Children's Hospital (LPCH) Vaccine Program during the 2010-2011 influenza season and received a single dose of TIV Fluzone (Sanofi Pasteur).

FIG. 20—Lack of correlation of plasma CXCL13 and Yellow Fever virus neutralizing antibody titer. FIG. 20A shows the correlation between the fold change in plasma CXCL13 seven days post immunization and yellow fever virus neutralizing antibody titer 14 days post immunization in 15 vaccinated individuals was not statistically significant. Yellow fever neutralizing antibody titers were determined using a standard neutralizing antibody assay (61) testing 2-fold dilutions of donor serum. Titer is reported as the last dilution with >50% Vero cell monolayer remaining.

FIG. 21—Lack of CXCL13 production by blood monocytes and blood CXCR5+ CD4 T cells. FIG. 21A shows monocytes gate by size [FSC (forward scatter)] and granularity [SSC (side scatter)] characteristics within human PBMCs. FIG. 21B show monocyte gated events from healthy donors or HIV+ individuals were either left unstimulated or stimulated for 4 h with PMA (phorbol 12-myristate 13-acetate) and Ionomycin in the presence of brefeldin A. Blood monocytes from both healthy and HIV+ donors produced TNF but did not produce CXCL13. FIG. 21C, (Upper) shows CD4+CXCR5+ gated or FIG. 21C, (Lower) CD4+CXC5+PD1+++ICOS+ gated blood cells were either left unstimulated or stimulated for 4 h with PMA and Ionomycin in the presence of brefeldin A. Both cell populations expressed CD40L after stimulation but did not produce CXCL13 in either condition. FIG. 21D shows frequency summary of CXCL13 producing cells from blood. Three healthy donor and three HIV+ donor samples were tested. GC Tfh cells from tonsil (from FIG. 14B) are included for comparison.

FIG. 22—Plasma CXCL13 is correlated with GC B-cell frequency. FIG. 22A shows human GC B cells as CD38+IgD—(gated on CD19+) B cells in human inguinal lymph node were matched with plasma CXCL13 samples in 14 human donors. Samples are color coded as in FIG. 14E: black, HIV+ antiretroviral-treated; blue, HIV seronegative; red, HIV+ viremic controller. The Spearman r and P values are shown. The GC B-cell percentage is plotted on log scale for visualization purposes only; the linear correlation results in an r2 value of 0.25. This panel corresponds to the GC Tfh-cell-CXCL13 correlation shown in FIG. 14E. FIG. 22B shows correlation of plasma CXCL13 and GC B cells (Bcl6+B220+) in naïve and alum+OVA immunized mice in FIG. 15C. GC B cells in naïve mice were set at the limit of detection (0.1% of total CD19+ B cells). FIG. 22C shows nonsignificant positive correlation of plasma CXCL13 and GC B cells (Ki67+Bcl6+CD20+) in the draining popliteal lymph node of macaques 7 d after the second or third protein and adjuvant immunization. Aluminum hydroxide or TLR (Toll-like receptor) ligand-encapsulated PLGA [poly(lactic-co-glycolic acid)] nanoparticles were used as adjuvants for gp140 Env and p55 Gag recombinant SIV (Simian immunodeficiency virus) proteins. This panel corresponds to the GC Tfh-cell-CXCL13 correlation shown in FIG. 16C.

FIG. 23 shows correlation of plasma CXCL13 concentration and Ab response after immunization. As in FIG. 17C except plasma CXCL13 concentration 7 d post immunization correlated with anti-gp140 (ConS; consensus group M) Env Ab responses (ELISA OD) 4 wk post immunization in 26 vaccinated individuals (HVTN068). Anti-gp140 Ab ELISA OD is background-subtracted.

### DESCRIPTION

Germinal centers (GC) perform the remarkable task of optimizing B cell antibody responses. GC are required for almost all B-cell receptor (BCR) affinity maturation and are an important parameter for monitoring for the production of neutralizing pathogen antibodies (e.g., HIV broadly neutralizing antibodies, HIV bnab) in response to an antigen or vaccination. However, lymphoid tissue is rarely available from immunized subjects, making the monitoring of GC activity via direct assessment of GC B cells and GC CD4+ T follicular helper cells (Tfh cells) problematic.

The CXCL13-CXCR5 chemokine axis plays a central role in organizing both B cell follicles and GCs, and GC Tfh cells can produce CXCL13 (C—X—C motif chemokine 13 (CXCL13), also known as B lymphocyte chemoattractant (BLC)) in the LN environment. As disclosed herein, systemic blood levels of CXCL13 correlate with germinal center activity and immune responses to antigens and vaccines. Non-limiting examples of indicators of GC activity include the number (i.e., amount) and/or size of germinal centers in a lymph node tissue, the number (i.e., amount) of CD4+ T cell follicular helper (Tfh) cells in a lymph node tissue, the number (i.e., amount) of Tfh cells in a lymph node tissue that produce CXCL13, and the number (i.e., amount) of circulating activated T-cells that are ICOS+, PD-1+, CXCR5+ and CD4+. Non-limiting examples of a lymph node tissue include mammalian lymph nodes of the head, neck, thorax, and limbs, including the tonsils, spleen and Peyer's patches.

For example, as shown herein, significantly increased levels of CXCL13 in the blood of a subject are associated with the generation of neutralizing antibodies against HIV in a large longitudinal cohort of HIV-infected individuals. An increase in blood CXCL13 levels were also shown to correlate with an increase in GC activity, as measured by an increase in one or more indicators of GC activity in immunized mice, immunized macaques, and in HIV-infected humans. Furthermore, blood CXCL13 levels in immunized humans correlated with an antibody response and ICOS+ Tfh-like cells in blood. Together, these findings support the use of CXCL13 as a plasma biomarker. In some embodiments, CXCL13 levels in blood can be used to monitor GC activity and immune responsiveness. For example CXCL13 levels can be used to monitor vaccine efficacy, antigen responses, the effectiveness of immunosuppressive drugs and efficacy of drugs for the treatment autoimmune disorders and/or allergy. In certain embodiments, CXCL13 levels can be monitored or used, alone or in combinations with other methods, to diagnose autoimmune disorders, allergy, immunosuppression, tolerance, certain types of blood born cancers and more. The invention therefore, in some embodiments, provides CXCL13 as a biomarker of germinal center activity and T cell follicular helper (Tfh) activity, and methods and uses thereof. In some embodiments, CXCL13 can be used as an indicator of vaccine-induced generation of broadly neutralizing antibodies (bnAb), for example for HIV vaccines. Since blood is a readily available sample, one can measure germinal center activity and immune responses from a subject's blood sample without requiring invasive collection of lymphoid tissue samples.

Molecules present in blood are of great interest to vaccine efforts as minimally invasive biomarkers for germinal center activity. CXCL13 is a ligand for CXCR5, and is an important chemokine for B cell migration and produced by GC Tfh cells. CXCL13 levels can be detected in human or macaque using anti-human CXCL13 antibodies that cross react with rhesus macaque CXCL13. CXCL13 can be detected by ELISA, or by use of another suitable immuno assay, in blood from rhesus macaques or human immunized with an immunogen or vaccine.

As shown herein, blood CXCL13 levels are increased one to two weeks post immunization compared to pre bleed baselines and plasma CXCL13 levels correlate with the percentage of GC Tfh cells found in draining lymph nodes. CXCL13 levels also correlated with the amount of activated ICOS+PD1+ CXCR5+ peripheral Tfh cells found circulating in the blood after immunization.

In certain embodiments, a presence or absence of an immune response of a subject may be determined or monitored by a method disclosed herein. The presence or absence of an immune response of a subject can be determine or monitor for any suitable reason. In some embodiments the immune response of a subject is determined or monitored after exposure of the subject to a pathogen. In some embodiments the immune response of a subject is determined or monitored after administration of an antigen, vaccine, drug or treatment to the subject. In some embodiments the immune response of a subject is determined or monitored for a subject having or suspected of having a disorder or condition (e.g., an autoimmune disorder), for example to diagnose a disorder or condition, or to monitor the progression of a disorder or condition.

Any suitable immune response can be monitored by a method herein. Non-limiting examples of an immune response in a subject include production of antigen specific antibodies, production of neutralizing antibodies (e.g., antibodies that neutralize a pathogen or toxin), production of antigen specific T-cells, production of antigen specific B-cells, an increase in GC activity, an increase in the number of GC in a lymph node tissue, an increase of Tfh cells in a lymph node tissue, and an increase of ICOS+PD1+CXCR5+CD4+ circulating T-cells.

In some embodiments an immune response includes production of antibodies by a subject in response to an administered antigen or vaccine, where the antibodies produced in response to the antigen or vaccine bind to a pathogen, or to a portion of a pathogen. A pathogen can be a virus, bacteria, fungus or parasite. In certain embodiments, an immune response includes production of neutralizing antibodies in response to an antigen or vaccine where the neutralizing antibodies can bind to a target pathogen and neutralize or kill the pathogen. Neutralizing antibodies can effectively block or inhibit one or more pathogenic functions (e.g., block or inhibit binding or entry of a pathogen to a host cell), neutralize or inactivate a toxin produced by a pathogen, and/or mediate killing of a pathogen.

In certain embodiments the presence of an immune response indicates the presence of circulating antibodies in a subject that bind specifically to an antigen, or to an antigen component of a vaccine (e.g., an antigen or vaccine that was administered to the subject). In some embodiments, the presence of an immune response indicates an amount of circulating antibodies in a subject after the administering an antigen or vaccine to the subject that is substantially larger than an amount of circulating antibodies in the subject prior to the administering of the antigen or vaccine, where the circulating antibodies bind specifically to the antigen, or to an antigen component of the vaccine that was administered.

In some embodiments, an immune response comprises production of antibodies in a subject in response to administration of an antigen, or a vaccine comprising the antigen, where the antibodies produced by the subject specifically bind to, and often bind with high affinity to, the antigen administered. An antibody that “specifically” binds to an antigen (e.g., antigen X) is an antibody that binds to antigen X in preference to binding another unrelated molecule or another peptide as determined by, for example, a suitable in vitro assay (e.g., an Elisa, Immunoblot, Flow cytometry, and the like). A specific binding interaction discriminates over non-specific binding interactions by having about 2-fold or more, often about 10-fold or more, and sometimes about 100-fold or more, 1000-fold or more, 10,000-fold or more, 100,000-fold or more, or 1,000,000-fold or more binding affinity for a specific antigen compared to other peptides or proteins. Methods of determining and quantitating specific binding are well known in the art. In certain embodiments, an effective immune response is determined according to the presence of, or an increase in, the amount (e.g., titer) of circulating antibodies in a subject that specifically bind to a particular antigen (e.g., an antigen component of a vaccine). In some embodiments, the absence of an immune response comprises a failure of a subject to produce antibodies that specifically bind to an antigen in response to administration of the antigen, or a vaccine comprising the antigen.

In certain embodiments, an antigen or vaccine is administered to a subject. Any suitable antigen or vaccine can be administered to a subject. In some embodiments an antigen is administered to a subject alone, with one or more different antigens, and/or with one or more adjuvants. In some embodiments, an antigen is any immunogenic molecule and can be derived from or isolated from any source (e.g., from any organism), synthesized chemically or produced by recombinant technology. Non-limiting examples of antigens include a peptide, protein, glycoprotein, carbohydrate, polysaccharide, lipid, nucleic acid, portion thereof, or combinations thereof. In some embodiments an antigen is produced by, or is native to a subject (e.g., a self-antigen); or is native to the species of a subject, but foreign to an individual subject (e.g., a human leukocyte antigen (HLA), or the like).

In some embodiments an antigen comprises one or more portions of a pathogen. Non-limiting examples of a pathogen include a virus, bacteria, fungus or parasite. An antigen can be any portion of a pathogen, non-limiting examples of which include a cell membrane or cell wall component; any peptide, protein, lipid, carbohydrate, or nucleic acid produced by a pathogen; any peptide or protein produced indirectly by a virus (e.g., a protein expressed from viral nucleic acid); a peptide or protein secreted by a pathogen (e.g., a toxin); a viral capsid, viral envelope, or viral coat protein, glycoprotein, or polysaccharide; the like, portions thereof of, or combinations thereof.

An antigen configured for safe administration to a subject with an expectation of inducing a protective immune response directed against a pathogen is often referred to as a vaccine. A vaccine may comprise any suitable antigen. In some embodiments a vaccine comprises one or more antigens. In some embodiments a vaccine comprises one or more suitable adjuvants. In some embodiments a vaccine comprises one or more pathogens (dead, alive, or attenuated), or portions thereof. For example, in some embodiments a vaccine comprises a live, dead or attenuated virus, bacteria, fungus or parasite. In some embodiments a vaccine is a commercially available and/or FDA approved vaccine. Commercially available vaccines are known and are contemplated for use herein, non-liming examples of which include Adenovirus Type 4 and Type 7 Vaccine, Anthrax Vaccine Adsorbed, BCG Live, Diphtheria & Tetanus Toxoids Adsorbed, Diphtheria & Tetanus Toxoids & Acellular Pertussis Vaccine Adsorbed, Hepatitis B (recombinant), Inactivated Poliovirus Vaccine, Inactivated Poliovirus and Haemophilus b Conjugate (Tetanus Toxoid Conjugate) Vaccine, Haemophilus b Conjugate Vaccine (Meningococcal Protein Conjugate), Haemophilus b Conjugate Vaccine (Tetanus Toxoid Conjugate), Haemophilus b Conjugate Vaccine (Meningococcal Protein Conjugate) & Hepatitis B Vaccine (Recombinant), Hepatitis A Vaccine, Inactivated, Human Papillomavirus Quadrivalent (Types 6, 11, 16, 18) Vaccine, Human Papillomavirus 9-valent Vaccine, Human Papillomavirus Bivalent (Types 16, 18) Vaccine, Influenza A (H1N1) 2009 Monovalent Vaccine, Influenza A (H1N1) 2009, Influenza Virus Vaccine, H5N1, Influenza Vaccine, Adjuvanted, Influenza Virus Vaccine (Trivalent, Types A and B), Influenza Vaccine, Live, Intranasal, Influenza Vaccine, Live, Intranasal (Quadrivalent, Types A and Types B), Japanese Encephalitis Virus Vaccine, Measles and Mumps Virus Vaccine, Meningococcal (Groups A, C, Y, and W-135) Oligosaccharide Diphtheria CRM197 Conjugate Vaccine, Meningococcal Groups C and Y and Haemophilus b Tetanus Toxoid Conjugate Vaccine, Meningococcal (Groups A, C, Y and W-135) Polysaccharide Diphtheria Toxoid Conjugate Vaccine, Meningococcal Group B Vaccine (BEXSERO), Meningococcal Group B Vaccine (TRUMENBA), Meningococcal Polysaccharide Vaccine, Groups A, C, Y and W-135 Combined, Plague Vaccine, Pneumococcal Vaccine, Pneumococcal 7-valent Conjugate Vaccine, Diphtheria CRM197 Protein, Pneumococcal 13-valent Conjugate Vaccine, Rabies Vaccine, Rotavirus Vaccine, Smallpox (Vaccinia) Vaccine, Typhoid Vaccine, Typhoid Vi Polysaccharide Vaccine, Varicella Virus Vaccine, Yellow Fever Vaccine, and Zoster Vaccine. In some embodiments a vaccine is an experimental vaccine.

In some embodiments, the absence or presence of an immune response is associated with the absence or presence of an increase in GC activity, respectively. GC activity can be detected, determine and/or monitored by a method herein. GC activity may increase for any number of reasons known in the art including, but not limited to disease progression, response to stimulus, response to a vaccine or antigen, response to an infection. In some embodiments, an increase in GC activity is in response to administration of a vaccine or antigen. In some embodiments, an increase in GC activity is in response to administration of a drug or treatment. In some embodiments, an increase in GC activity is due to progression of a disease or disorder (e.g., an autoimmune disorder). Thus the presence of an immune response often comprises an increase in GC activity.

In certain embodiments an increase in GC activity refers to an increase in the number or amount of GCs in one or more lymph node tissues of a subject. Thus, in some embodiments, an increase in GC activity comprises an increase in the number of GCs in one or more lymph node tissues in a subject. In some embodiments, an increase in the number of GCs in one or more lymph node tissues is at least a 10% increase in the number of GCs in one or more lymph node tissues in a subject. In some embodiments, an increase in the number of GCs in one or more lymph node tissues of a subject is at least a 30% increase, at least a 50% increase or at least a 75% increase in the number of GC in one or more lymph node tissues of a subject. In certain embodiments, an increase in the number of GCs in one or more lymph node tissues of a subject is at least a 2-fold, at least a 5-fold or at least a 10-fold increase in the number of GCs in one or more lymph node tissues. An increase in GCs is often determined by comparing the amount of GCs in one or more lymph node tissues at some first time point to the amount of GCs in the one or more lymph node tissues at some later time point. In certain embodiments, an increase in the amount of GCs is often determined by comparing the amount of GCs in one or more lymph node tissues before administration of a drug, antigen or vaccine, to an amount of GCs in one or more lymph node tissues after administration of a drug, antigen or vaccine. In some embodiments, an presence or absence of an increase in the amount of GCs in a subject is detected or determined by a method disclosed herein, for example by determining the presence or absence of an increase in an amount of CXCL13 in the blood of a subject. In certain embodiments an absence of GC activity refers to the absence of an increase in the number of GCs in one or more lymph node tissues of a subject.

In certain embodiments an increase in GC activity refers to an increase in the number, activity, or amount of Tfh cells in one or more lymph node tissues of a subject. Thus, in some embodiments, an increase in GC activity comprises an increase in the number or amount of Tfh cells in one or more lymph node tissues in a subject. In some embodiments, an increase in the number of Tfh cells in one or more lymph node tissues is at least a 10% increase in the number of Tfh cells in one or more lymph node tissues in a subject. In some embodiments, an increase in the number of Tfh cells in one or more lymph node tissues of a subject is at least a 30% increase, at least a 50% increase or at least a 75% increase in the number of Tfh cells in one or more lymph node tissues of a subject. In certain embodiments, an increase in the number of Tfh cells in one or more lymph node tissues of a subject is at least a 2-fold, at least a 5-fold or at least a 10-fold increase in the number of Tfh cells in one or more lymph node tissues. An increase in Tfh cells is often determined by comparing the amount of Tfh cells in one or more lymph node tissues at some first time point to the amount of Tfh cells in the one or more lymph node tissues at some later time point. In certain embodiments, an increase in the amount of Tfh cells is often determined by comparing the amount of Tfh cells in one or more lymph node tissues before administration of a drug, antigen or vaccine, to an amount of Tfh cells in one or more lymph node tissues after administration of a drug, antigen or vaccine. In some embodiments, a presence or absence of an increase in the amount of Tfh cells in a subject is detected or determined by a method disclosed herein, for example by determining the presence or absence of an increase in an amount of CXCL13 in the blood of a subject. In certain embodiments an absence of GC activity refers to the absence of an increase in the number of Tfh cells in one or more lymph node tissues of a subject.

Tfh cells can produce and or secrete CXCL13. In certain embodiments an increase in GC activity refers to an increase in the number, activity, or amount of Tfh cells that produce or secrete CXCL13 in one or more lymph node tissues of a subject. Thus, in some embodiments, an increase in GC activity comprises an increase in the number or amount of Tfh cells that produce or secrete CXCL13 in one or more lymph node tissues in a subject. In some embodiments, an increase in the number of Tfh cells that produce or secrete CXCL13 in one or more lymph node tissues is at least a 10% increase in the number of Tfh cells that produce or secrete CXCL13 in one or more lymph node tissues in a subject. In some embodiments, an increase in the number of Tfh cells that produce or secrete CXCL13 in one or more lymph node tissues of a subject is at least a 30% increase, at least a 50% increase or at least a 75% increase in the number of Tfh cells that produce or secrete CXCL13 in one or more lymph node tissues of a subject. In certain embodiments, an increase in the number of Tfh cells that produce or secrete CXCL13 in one or more lymph node tissues of a subject is at least a 2-fold, at least a 5-fold or at least a 10-fold increase in the number of Tfh cells that produce or secrete CXCL13 in one or more lymph node tissues. An increase in Tfh cells that produce or secrete CXCL13 is often determined by comparing the amount of Tfh cells that produce or secrete CXCL13 in one or more lymph node tissues at some first time point to the amount of Tfh cells that produce or secrete CXCL13 in the one or more lymph node tissues at some later time point. In certain embodiments, an increase in the amount of Tfh cells that produce or secrete CXCL13 is often determined by comparing the amount of Tfh cells that produce or secrete CXCL13 in one or more lymph node tissues before administration of a drug, antigen or vaccine, to an amount of Tfh cells that produce or secrete CXCL13 in one or more lymph node tissues after administration of a drug, antigen or vaccine. In some embodiments, a presence or absence of an increase in the amount of Tfh cells that produce or secrete CXCL13 in a subject is detected or determined by a method disclosed herein, for example by determining the presence or absence of an increase in an amount of CXCL13 in the blood of a subject. In certain embodiments an absence of GC activity refers to the absence of an increase in the number of Tfh cells that produce or secrete CXCL13 in one or more lymph node tissues of a subject.

In certain embodiments an increase in GC activity refers to an increase in the number or amount of circulating activated T-cells in the subject, where the activated T-cells are ICOS+PD1+CXCR5+CD4+(i.e., activated ICOS+PD1+CXCR5+CD4+ T-cells) in a subject. Thus, in some embodiments, an increase in GC activity comprises an increase in the number or amount of activated ICOS+PD1+CXCR5+CD4+ T-cells in the blood of a subject (circulating activated ICOS+PD1+CXCR5+CD4+ T-cells). In some embodiments, an increase in the number of circulating activated ICOS+PD1+CXCR5+CD4+ T-cells in a subject is at least a 10% increase in the number of circulating activated ICOS+PD1+CXCR5+CD4+ T-cells in a subject. In some embodiments, an increase in the number of circulating activated ICOS+PD1+CXCR5+CD4+ T-cells is at least a 30% increase, at least a 50% increase or at least a 75% increase in the number of circulating activated ICOS+PD1+CXCR5+CD4+ T-cells in a subject. In certain embodiments, an increase in the number of circulating activated ICOS+PD1+CXCR5+CD4+ T-cells in a subject is at least a 2-fold, at least a 5-fold or at least a 10-fold increase in the number of circulating activated ICOS+PD1+CXCR5+CD4+ T-cells in a subject. An increase in circulating activated ICOS+PD1+CXCR5+CD4+ T-cells is often determined by comparing the amount of circulating activated ICOS+PD1+CXCR5+CD4+ T-cells in a subject at some first time point, to an amount of circulating activated ICOS+PD1+CXCR5+CD4+ T-cells in the subject at some later time point. In certain embodiments, an increase in the amount of circulating activated ICOS+PD1+CXCR5+CD4+ T-cells is often determined by comparing the amount of circulating activated ICOS+PD1+CXCR5+CD4+ T-cells before administration of a drug, antigen or vaccine, to an amount of circulating activated ICOS+PD1+CXCR5+CD4+ T-cells in a subject after administration of a drug, antigen or vaccine. In some embodiments, a presence or absence of an increase in the amount of circulating activated ICOS+PD1+CXCR5+CD4+ T-cells in a subject is detected or determined by a method disclosed herein, for example by determining the presence or absence of an increase in an amount of CXCL13 in the blood of a subject. In certain embodiments an absence of GC activity refers to the absence of an increase in the number of circulating activated ICOS+PD1+CXCR5+CD4+ T-cells in a subject.

In some embodiments, a change in GC activity is detected or determined by a method disclosed herein. In some embodiment, GC activity is an indicator of disease progression of an autoimmune disorder, where an increase in GC activity indicates progression of the disease. Thus, in certain embodiments, GC activity can be detected or monitored by a method herein to monitor the efficacy of a drug administered for the treatment of an autoimmune disorder. In some embodiment, GC activity is an indicator of HIV progression, where a decrease in GC activity indicates progression of the disease. Thus, in certain embodiments, GC activity can be detected or monitored by a method herein to monitor the efficacy of a drug administered for the treatment of HIV. In some embodiment, GC activity is an indicator of immunosuppression induced by chemotherapy, radiation, or immunosuppressive drugs (e.g., administered to transplant recipients), where a decrease in GC activity indicates a suppression of a subject's immune system. Thus, in certain embodiments, GC activity can be detected or monitored by a method herein to monitor immunosuppression. Therefore, in certain embodiments a method comprises determining a first amount of CXCL13 in a first sample obtained from a subject, determining a second amount of CXCL13 in a second sample obtained from a subject, wherein the second sample is obtained at least 1 week after the first sample is obtained, comparing the first and second amount of CXCL13, wherein an increase in the second amount of CXCL13 compared to the first amount of CXCL13 indicates an increase in GC activity, a decrease in the second amount of CXCL13 compared to the first amount of CXCL13 indicates a decrease in GC activity, and the absence of a change in the second amount of CXCL13 compared to the first amount indicated no significant change in GC activity in the subject. In certain embodiments a method comprises determining a first amount of CXCL13 in a first sample obtained from a subject, administering a drug or treatment to the subject, determining a second amount of CXCL13 in a second sample obtained from a subject, wherein the second sample is obtained at least 1 week after the first sample is obtained, comparing the first and second amount of CXCL13, wherein an increase in the second amount of CXCL13 compared to the first amount of CXCL13 indicates an increase in GC activity, a decrease in the second amount of CXCL13 compared to the first amount of CXCL13 indicates a decrease in GC activity, and the absence of a change in the second amount of CXCL13 compared to the first amount indicated no significant change in GC activity in the subject.

Any suitable drug or treatment can be administered to a patient. In certain embodiments a drug comprises an antiviral used for treating an HIV infection in a subject. In some embodiments, a drug comprises an immunosuppressive drug used to treat a transplant recipient. In some embodiments a treatment comprises radiation treatment and/or chemotherapy.

The amount of CXCL13 in one or more samples may be determined by any suitable method, non-limiting examples of which include an immunoassay (e.g., an ELISA, Flow Cytometry (e.g., a bead or particle platform, e.g., a CBA-based assay, e.g., Luminex and the like), Western Blot, ELISPOT, immunochromatographic stick, etc.), Mass Spectrometry (MS), LC/MS, High-performance liquid chromatography (HPLC), any suitable separation technique, the like, and combinations thereof. In some embodiments, an amount of CXCL13 is determined by use of a binding agent that specifically binds to CXCL13. Non-limiting examples of binding agents include an antibody (e.g., obtained or made by any suitable method, or obtained from any suitable source), TandAbs, nanobodies, BiTEs, SMIPs, DARPins, DNLs, affibodies, Duocalins, adnectins, fynomers, Kunitz Domains Albu-dabs, DARTs, DVD-IG, Covx-bodies, peptibodies, scFv-Igs, SVD-Igs, dAb-Igs, Knob-in-Holes, triomAbs, the like, and binding portions thereof. In some embodiments, an amount of CXCL13 is a concentration of CXCL13 (e.g., a concentration of CXCL13 in a sample). In certain embodiments, an amount of CXCL13 is a relative amount.

In some embodiments an amount of CXCL13 is not determined by a nucleic acid assay. Non-limiting examples of nucleic acid assays detect an amount of DNA or RNA (e.g., mRNA) to determine an amount of CXCL13. In some embodiments an amount of CXCL13 is not determined by analysis of whole cells. Thus, in some embodiments, an amount of CXCL13 is determined by a method that consists essentially of an immuno assay that utilizes a binding agent that binds specifically to CXCL13, for example, where an amount of CXCL13 is not determined by a nucleic acid assay or by analysis of whole or lysed cells (e.g., cell cytoplasm or cell membranes). In some embodiments an amount of CXCL13 that is determined or detected by a method disclosed herein is an amount of extracellular, soluble CXCL13 found in blood or a blood product of a subject.

In some embodiments the amounts (e.g., absolute, average, mean or relative amounts) of CXCL13 in two or more samples are compared. Any suitable method can be used for comparing amounts of CXCL13 in two or more samples can be used. In some embodiments a comparison comprises comparing a first amount of CXCL13 in a sample to a second amount of CXCL13 in a sample. A comparison often provides an output. An output is sometimes a ratio (e.g., a ratio of a first amount of CXCL13 to a second amount of CXCL13. In certain embodiments an output is a determination that two values (e.g., a first and second amount of CXCL13) are significantly different. In certain embodiments, a significant difference is a significant increase or a significant decrease. For example, a second amount of CXCL13 may be significantly greater than or less than a first amount of CXCL13. Thus, in some embodiments, a comparison may provide a determination that a second amount of CXCL13 is significantly greater than a first amount of CXCL13, which comparison indicates the presence of an increase (e.g., an increase, e.g., a significant increase) in the second amount of CXCL13 (e.g., where the second sample is obtained after the first sample). In some embodiments, a comparison may provide a determination that a second amount of CXCL13 is not significantly greater than a first amount of CXCL13, which comparison (e.g., output) indicates the absence of an increase (e.g., absence of a significant increase) in the second amount (e.g., where the second sample is obtained after the first sample). In some embodiments, a comparison may provide a determination that a first amount of CXCL13 is significantly greater than a second amount of CXCL13, which comparison indicates the presence of a decrease (e.g., a decrease, e.g., a significant decrease) in the second amount of CXCL13 (e.g., where the second sample is obtained after the first sample). In some embodiments, a comparison may provide a determination that a first amount of CXCL13 is not significantly different than a second amount of CXCL13, which comparison indicates the absence of an increase or a decrease (e.g., no change, e.g., no significant change) in the second amount of CXCL13.

In some embodiments, a significant difference (e.g., a second amount that is significantly greater than a first amount, or vice versa) is determined according to a significant difference between the two amounts. In some embodiments, a significant difference refers to a statistical difference or a statistically significant difference. A statistically significant difference is sometimes a statistical assessment of an observed difference. A statistically significant difference can be assessed by a suitable method in the art. Any suitable threshold or range can be used to determine that two amounts are significantly different. In some cases two amounts (e.g., average or mean amounts) that differ by about 0.01 percent or more are significantly different. In some cases, two amounts that differ by about 0.5 percent or more are significantly different. Sometimes two amounts that differ by about 1%, 2%, 5%, 7% or more than about 10% are significantly different. Sometimes two amounts are significantly different and there is no overlap in either amount and/or no overlap in a range defined by an uncertainty value calculated for one or both amounts. Non-limiting examples of an uncertainty value include a standard deviation, standard error, calculated variance, p-value, and mean absolute deviation (MAD). In some cases an uncertainty value is a standard deviation expressed as sigma. Sometimes two amounts are significantly different and they differ by about 1 or more times the uncertainty value (e.g., 1 sigma). Sometimes two amounts are significantly different and they differ by about 2 or more times the uncertainty value (e.g., 2 sigma), about 3 or more, about 4 or more, about 5 or more, about 6 or more, about 7 or more, about 8 or more, about 9 or more, or about 10 or more times the uncertainty value.

Thus, in some embodiments, the presence of an increase (e.g., an increase) in a second amount of CXCL13 compared to a first amount of CXCL13 is a second amount that is greater than and significantly different than (i.e., significantly greater than) the first amount (e.g., where the second sample is obtained after the first sample). Likewise, in some embodiments, the absence of an increase in a second amount of CXCL13 compared to a first amount of CXCL13 (e.g., where the second sample is obtained after the first sample) means (i) there is no significant difference between the second amount and the first amount, or (ii) the first amount is significantly greater than the second amount. In some embodiments, a decrease in the second amount of CXCL13 (e.g., where the second sample is obtained after the first sample) is a second amount that is less than, and significantly different than (e.g., significantly less than) a first amount of CXCL13.

In certain embodiments, the presence of an increase (e.g., an increase) in a second amount compared to a first amount of CXCL13 (e.g., where the second sample is obtained after the first sample) comprises a second amount that is at least 10%, at least 15%, at least 20%, at least 25%, at least 30%, at least 50%, or at least 75% greater than a first amount. A second amount that is at least 10%, at least 15%, at least 20%, at least 25%, at least 30%, at least 50%, or at least 75% greater than a first amount indicates a respective increase in the second amount (e.g., where the second sample is obtained after the first sample). For example, a second amount of CXCL13 that is at least 10% greater than a first amount of CXCL13 indicates at least a 10% increase in the amount of CXCL13 (e.g., where the second sample is obtained after the first sample).

In certain embodiments, the presence of an increase in a second amount compared to a first amount of CXCL13 (e.g., where the second sample is obtained after the first sample) comprises a second amount that is at least 2-fold greater, at least 3-fold greater, at least 4-fold greater, at least 5-fold greater, at least 10-fold greater, or at least 100-fold greater than a first amount. A second amount that is at least 12-fold greater, at least 3-fold greater, at least 4-fold greater, at least 5-fold greater, at least 10-fold greater, or at least 100-fold greater than a first amount indicates a respective increase in the second amount (e.g., where the second sample is obtained after the first sample). For example, a second amount of CXCL13 that is at least 2-fold greater than a first amount of CXCL13 indicates at least a 2-fold increase in the amount of CXCL13 (e.g., where the second sample is obtained after the first sample).

In certain embodiments, a decrease in a second amount compared to a first amount of CXCL13 (e.g., where the second sample is obtained after the first sample) comprises a second amount that is at least 10%, at least 15%, at least 20%, at least 25%, at least 30%, at least 50%, or at least 75% less than a first amount. A second amount that is at least 10%, at least 15%, at least 20%, at least 25%, at least 30%, at least 50%, or at least 75% less than a first amount indicates a respective decrease in the second amount (e.g., where the second sample is obtained after the first sample). For example, a second amount of CXCL13 that is at least 10% less than a first amount of CXCL13 indicates at least a 10% decrease in the amount of CXCL13 (e.g., where the second sample is obtained after the first sample).

In certain embodiments, decrease in a second amount compared to a first amount of CXCL13 (e.g., where the second sample is obtained after the first sample) comprises a second amount that is at least 2-fold less, at least 3-fold less, at least 4-fold less, at least 5-fold less, at least 10-fold less, or at least 100-fold less than a first amount. A second amount that is at least 2-fold less, at least 3-fold less, at least 4-fold less, at least 5-fold less, at least 10-fold less, or at least 100-fold less than a first amount indicates a respective decrease in the second amount (e.g., where the second sample is obtained after the first sample). For example, a second amount of CXCL13 that is at least 2-fold less than a first amount of CXCL13 indicates at least a 2-fold decrease in the amount of CXCL13 (e.g., where the second sample is obtained after the first sample).

In certain embodiments the presence of an increase in a second amount of CXCL13 compared to a first amount of CXCL13 (e.g., where the second sample is obtained after the first sample) indicates the presence of an immune response. In certain embodiments the absence of an increase in a second amount of CXCL13 compared to a first amount of CXCL13 (e.g., where the second sample is obtained after the first sample) indicates the absence of an immune response. In some embodiments, the absence of an immune response indicates a subject is not responsive to an antigen or vaccine. In some embodiments, the absence of an immune response indicates a subject is immunosuppressed or immune-incompetent.

The term “subject” refers to animals, typically mammalian animals. Any suitable mammal can be used for a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Thus, in certain embodiments, a subject is a mammal. In some embodiments a mammal is a human. In some embodiments, a subject is a human. A subject can be any age or at any stage of development (e.g., an adult, teen, child, infant, or a mammal in utero). A subject can be male or female. A subject can be a pregnant female. In certain embodiments a subject can be an animal disease model, for example, animal models used for the study of viral infections.

In some embodiments a subject or mammal has, or is suspected of having an autoimmune disorder. In some embodiments a subject does not have an autoimmune disorder. In some embodiments a subject or mammal has, or is suspected of having an infection. For example a subject has, or is suspected of having a bacterial, fungal, parasitic or viral infection. In some embodiments a subject does not have an infection. In some embodiments a subject or mammal is “at risk” of an infection (e.g., a bacterial, fungal, parasitic or viral infection). A mammal that is at risk may have increased risk factors for an infection, non-limiting examples of which include immunocompromised individuals or immune deficient subjects (e.g., bone marrow transplant recipients, irradiated individuals, subjects having certain types of cancers, particularly those of the bone marrow and blood cells (e.g., leukemia, lymphoma, multiple myeloma), subjects with certain types of chronic infections (e.g., HIV, e.g., AIDS), subjects treated with immunosuppressive agents, subjects suffering from malnutrition and aging, subjects taking certain medications (e.g. disease-modifying anti-rheumatic drugs, immunosuppressive drugs, glucocorticoids) and subjects undergoing chemotherapy), the like or combinations thereof). In certain embodiments, a subject is immunocompromised and/or immune deficient. In some embodiments a subject at risk is, will be, or has been in a location or environment suspected of containing a virus (e.g., filovirus). For example, a subject at risk can be a medical professional that is providing care to another who is suspected of being infected with, or known to be infected with a virus (e.g., a filovirus). In certain embodiments, a subject at risk is any subject that has been exposed to a virus (e.g., a filovirus).

A sample may be obtained from a subject. In some embodiments a sample is a blood sample, or a tissue sample from which blood, or a blood product can be obtained. For example, blood and/or tissue can be obtained from a subject, then processed by a method known in the art to obtain a blood product suitable for testing by a method disclosed herein. Non-limiting examples of blood products include whole blood, plasma, serum, lymphatic fluid, cerebral spinal fluid and buffy coats. In some embodiments a sample comprises blood, or a blood product (e.g., plasma, serum, and the like). In certain embodiments, a sample consists essentially of blood or a blood product. A sample that consists essentially of blood or a blood product may include other components that were not derived from a subject such as reagents, buffers, substrates, anti-coagulants (e.g., heparin), binding agents, salts, and the like. In some embodiments a sample consists essentially of serum and/or plasma obtained from a subject. In some embodiments a sample consists essentially of cerebral spinal fluid.

In some embodiments one or more samples are obtained or isolated from the same subject. For example, a first sample and a second sample may be obtained from the same subject. In some embodiments two or more samples are obtained or isolated from different subjects. In certain embodiments, two or more samples are obtained from different species. For example, in some embodiments, a first sample is a suitable control sample. A control sample is often a standard, or provides a standard amount of CXCL13 used as a base level for comparison. In some embodiments a standard comprises an amount of CXCL13 that represents an amount (absolute, average, or mean amount) of CXCL13 found in a healthy subject who is devoid of any autoimmune disorders, and/or who is not having, or has not recently had an immune response to a vaccine, pathogen or antigen. A control sample may be obtained from any suitable species. A control sample may be obtained from a control subject, or from a control group. For example, in certain embodiments, a first sample can be a control sample obtained from a mixture of samples for the purpose of providing a standard or control. In some embodiments a control sample (e.g., a first sample) comprises a known amount of CXCL13. In some embodiments a control sample (e.g., a first sample) is not obtained from a subject and contains a known amount of a recombinant or synthesized CXCL13. In some embodiments a control sample is two or more samples used for the purpose of generating a standard curve.

A sample may be obtained from a subject by any suitable means. In some embodiments a sample is obtained by a care giver or a medical professional. In some embodiments a sample is obtained by the subject. A sample obtained from a subject can be used directly for a method herein. In some embodiments, a sample is obtained from a subject and provided (e.g., by a third party) for use in a method disclosed herein. For example, in certain embodiments, a sample obtained from a subject may be extracted from a subject by a medical professional, sent to a first lab for processing and optionally provided to another for use in a method disclosed herein.

One or more samples also may be isolated at different time points as compared to another sample, where each of the samples are from the same subject. For example a first sample can be obtained from a subject at a first suitable time point and a second sample may be obtained from the subject at a later suitable time point. In some embodiments, a first sample and a second sample are obtained from a subject where the second sample is obtained or isolated from a subject at least 1 week, at least 2 weeks, at least 3 weeks, at least 4 weeks, at least 5 weeks, at least 10 week or at least 15 weeks after obtaining or isolating the first sample from the subject. In some embodiments, a first sample and a second sample are obtained from a subject where the first sample is obtained or isolated from the subject at a suitable time prior to administering a drug, treatment, antigen or vaccine to the subject and the second sample is obtained or isolated from the subject at a suitable time after administering a drug, treatment, antigen or vaccine to the subject. A first sample may be obtained or isolated from a subject at anytime from 5 years to 1 minute prior to, from 1 year to 1 minute prior to, from 6 months to 1 minute prior to, from 1 month to 1 minute prior to, from 4 weeks to 1 minute prior to or from 2 weeks to 1 minute prior to administering a drug, treatment, antigen or vaccine to the subject. In some embodiments, a first sample and a second sample are obtained from a subject where the second sample is obtained or isolated from a subject at least 1 week, at least 2 weeks, at least 3 weeks, at least 4 weeks, at least 5 weeks, at least 10 week or at least 15 weeks after administering a drug, treatment, antigen or vaccine to the subject. In some embodiments, a second sample is obtained from a subject at any time from about 1 day to about 20 weeks, from about 1 day to 15 weeks, from about 5 days to about 15 weeks, from about 1 week to about 15 weeks, or from about 1 week to about 5 weeks after administering a drug, treatment, antigen or vaccine to the subject.

In some embodiments a subject in need of a treatment or a composition described herein is a subject having, suspected of having or at risk of having an autoimmune disorder. In some embodiments a subject has or is suspected of having an autoimmune disorder. In certain embodiments, an autoimmune disorder is characterized by the presence of autoantibodies. Non-limiting examples of autoimmune disorders include, polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-oesophageal reflux disease, erythroderma, Behçet's disease, amyotrophic lateral sclerosis, transplantation, Neuromyelitis Optica, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility or immune-mediated infertility.

In some embodiment a method comprises determining a first amount of CXCL13 in a sample obtained from a subject; administering a suitable drug or treatment to the subject, wherein the drug or treatment is for the treatment of an autoimmune disorder; and determining a second amount of CXCL13 in a second sample; and comparing the first amount of CXCL13 to the second amount of CXCL13. In certain embodiments the drug or treatment administered to the subject is discontinued, continued, increased or decreased according to the comparison. For example, in same subjects having an autoimmune disorder, GC activity is relatively high compared to a normal healthy subject that does not have an autoimmune disorder. Therefore the goal of a treatment is to decrease GC activity in the patient. Accordingly, in certain embodiments, where the second amount of CXCL13 (i.e., after treatment) is less than a first amount of CXCL13 (i.e., before treatment), a treatment is often determined to be effective at treating the disorder, and in some embodiments the treatment is continued. In certain embodiments, where the second amount of CXCL13 (i.e., after treatment) is greater than, or the same as (i.e., not significantly different), a first amount of CXCL13 (i.e., before treatment), a treatment is often not effective, and in some embodiments the treatment is discontinued, or the amount of treatment is increased (e.g., a dose of an autoimmune drug may be increased).

In some embodiments a method comprises diagnosing a subject with an autoimmune disorder. In certain embodiments the method comprises providing a first amount of CXCL13 in a first sample, determining a second amount of CXCL13 in a second sample, where the second sample is obtained from the subject, and comparing the first amount of CXCL13 to the second amount, where a diagnosis of an autoimmune disorder is determined according to the comparison. In some embodiments the first sample is a control sample or standard sample. In some embodiments where the second amount of CXCL13 in greater than (e.g., at least 30% greater than) the first amount of CXCL13 in the first sample, the subject is determined to have an autoimmune disorder.

The exact formulation and route of administration for a composition described herein (e.g., a drug, antigen, vaccine) can be chosen by the individual physician in view of the patient's condition. See e.g., Fingl et al. 1975, in “The Pharmacological Basis of Therapeutics,” Ch. 1 p. 1; which is incorporated herein by reference in its entirety. Any suitable route of administration can be used for administration of a composition described herein. Non-limiting examples of routes of administration include topical or local (e.g., transdermally or cutaneously, (e.g., on the skin or epidermus), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranially, intraarticular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is often provided to a subject for self-administration or for administration to a subject by another (e.g., a non-medical professional). For example a composition described herein can be provided as an instruction written by a medical practitioner that authorizes a patient to be provided a composition or treatment described herein (e.g., a prescription). In another example, a composition can be provided to a subject wherein the subject self-administers a composition orally, intravenously or by way of an inhaler, for example.

Certain embodiments provide pharmaceutical compositions (e.g., drugs, antigens and vaccines) suitable for use in the technology, which include compositions where the active ingredients or antigens are contained in an amount effective to achieve its intended purpose. A “therapeutically effective amount” means an amount to prevent, treat, reduce the severity of, delay the onset of, or inhibit a symptom of an autoimmune disorder or pathogen infection, for example. A symptom can be a symptom already occurring or expected to occur. Determination of a therapeutically effective amount is well within the capability of those skilled in the art, especially in light of the detailed disclosure provided herein.

In other embodiments, a therapeutically effective amount can describe the amount necessary for a significant quantity of the composition to contact a desired region or tissue.

### EXAMPLES

### Example 1—CXCL13 is a Plasma Biomarker of Germinal Center Activity

Observation of plasma biomarkers allows for the monitoring of biological processes and disease states occurring in less accessible tissues with minimal intrusion and reduced costs. Biomarkers for many diseases, such as cancer, autoimmunity, and infection are currently under investigation Immunological evaluation of responses to vaccination in humans relies heavily on analysis of circulating antibodies and T cells. Generally, little is known about the status of the immune response in the lymph node(s) that drain the site of immunization in vaccinated humans. As lymphoid tissue is rarely available during a clinical vaccine trial, the present inventor set out to find blood biomarkers of the lymph node response.

The germinal center (GC) reaction is a critical immunological process that occurs in the draining lymph nodes after immunization (1). The GC response consists of antigen-specific B cells undergoing affinity maturation through a process of somatic hypermutation (SHM) of the B cell receptor (BCR). SHM is necessary for producing high affinity antibody responses after immunizations and infections. Particularly high levels of SHM, 15-30% mutation of the BCR amino acid sequence (2-5), are present and necessary for broad antibody neutralization of diverse HIV strains (6, 7). As candidate HIV vaccines are evaluated for the ability to induce broadly neutralizing antibodies, the quantitation and functional characterization of GC responses will be a key parameter for study (8).

Central to the GC reaction and SHM is the interaction of GC B cells with germinal center T follicular helper cells (GC Tfh) (9-11). GC Tfh cells are not only required for the GC reaction to occur, but are also limiting for the size of the reaction (9). In addition, GC Tfh have been shown to control the number of GC B cell divisions and therefore the amount of SHM by individual GC B cell clones (12). GC Tfh cells alternatively instruct GC B cells to undergo differentiation into memory B cells or plasma cells, continue to proliferate as GC B cells, or die by apoptosis (13). Currently, the preferred means of quantifying the GC response is the cellular enumeration and analysis of GC Tfh and GC B cells (14). However, in human and non-human primate (NHP) vaccination studies, direct analysis of draining lymph node tissue is impractical, invasive and/or undesirable for fear of disturbing the ongoing immune response.

The CXCL13-CXCR5 chemokine axis plays a major role in organizing both B cell follicles and GCs (15-17). CXCL13 is expressed by both follicular dendritic cells (18) and GC Tfh cells (19, 20) in the B cell follicles. B cell expression of CXCR5, the receptor for CXCL13, is necessary for migration to the follicle. Likewise, Tfh cells express CXCR5 to migrate to the border between the T cell zone and the B cell follicle then further upregulate CXCR5 to enter the GC. In addition to its chemoattractant properties necessary for normal lymphoid tissue organization, CXCL13 signaling in B cells has cytokine like properties important for adhesion (21), lymphotoxin synthesis (15), and potentially survival (22). And while CXCL13 acts locally it can also be detected in human plasma in the steady state. Furthermore, perturbations in plasma CXCL13 have been detected during chronic HIV and HCV infections (23, 24), various autoimmune diseases (25, 26), and metastatic breast cancer (27).

As GC Tfh regulate the size of the GC response and can be major producers of CXCL13, the present inventor explored whether plasma CXCL13 may reflect lymphoid tissue GC activity.

### Results

Plasma CXCL13 is Elevated in HIV⁺ bnAb Individuals

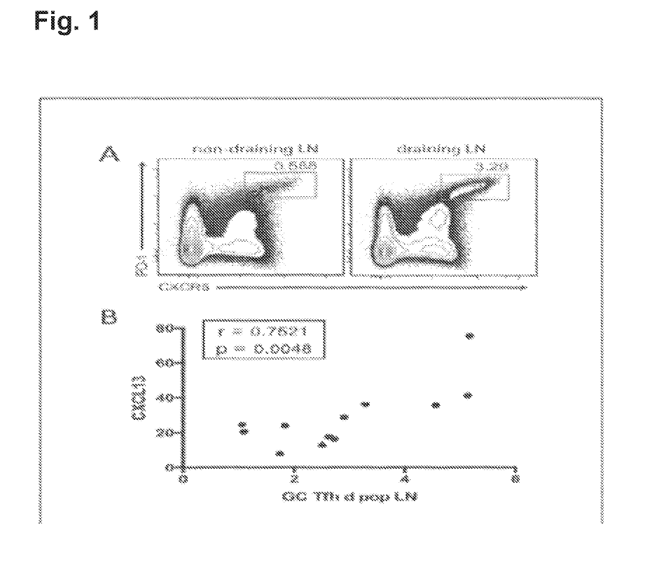

The high levels of somatic hypermutation seen in bnAbs generated against HIV (5, 28) and the association of circulating memory Tfh cells with the generation of bnAb (29) suggests that individuals able to generate HIV bnAb may have superior GC responses (8). Since directly measuring these tissue resident GC responses in humans is not generally feasible, the present inventor asked whether CXCL13 was higher in the plasma of individuals able to generate HIV bnAb than individuals that were not. Plasma samples from ART-naive HIV+ individuals enrolled in IAVI Protocol C were tested; a large longitudinal cohort of HIV+ individuals, monitored early after infection and followed every 3-6 months. This cohort has been extensively characterized for the ability of each individual to produce neutralizing antibodies against HIV ((29); Landais and Poignard, unpublished). A neutralization score was calculated based on both the breadth and potency of the nAb present in individual samples for each time point after infection. Fifteen percent of the 228 individuals followed beyond 4 years post infection were found to have a neutralization score of greater than 1 and were termed top neutralizers (FIG. 1A). Top neutralizers had neutralizing Ab responses capable of neutralizing an average of 27 of 37 (73%) pseudoviruses of a principally Tier 2 virus panel. The majority of HIV infected individuals had a neutralization score of less than 0.5, neutralizing an average of 10 of 37 (27%) pseudoviruses; these individuals were termed low neutralizers. Low neutralizers had either highly strain specific Tier 2 neutralizing Abs (almost certainly restricted to sequences closely related to their autologous infecting virus) or no measurable Tier 2 neutralizing antibodies. Plasma was tested for neutralizing Abs and CXCL13 concentration at both the earliest time point available after infection (˜4 months post infection) and at the time of bnAb development (˜40 months post infection). Broad neutralization was associated with 2-fold higher levels of plasma CXCL13 at both the ˜4 month (top neutralizers median: 92.7 pg/ml vs low neutralizers median: 31.3 pg/ml, p=0.0121) and the ˜40 month (top neutralizers median: 78.9 pg/ml vs low neutralizers median: 32.2 pg/ml p=0.0077) time points post infection (FIGS. 1B and C). The generation of HIV neutralizing antibodies is also positively correlated with HIV viral load (30) and viral load could affect plasma CXCL13 levels (31, 32). Viral loads were higher in the top neutralizer group at both time points tested (Figure S1). The present inventor therefore asked whether viral load and CXCL13 were independent variables. The difference in plasma CXCL13 between top and low neutralizers remained significant (ANCOVA, p=0.0205) at the ˜40 month time point and continued to show a strong trend at the ˜4 month time point (ANCOVA, p=0.0658). Higher viral loads are associated with higher plasma CXCL13 and may influence plasma CXCL13 (32). However, our analysis shows that plasma CXCL13 and viral load are largely independent factors each correlated with the generation of neutralizing antibodies to HIV. Therefore, elevated plasma CXCL13 in top neutralizers suggested that these individuals may have more active GC responses.

Plasma CXCL13 is Correlated with Lymphoid Tissue Germinal Centers in Humans

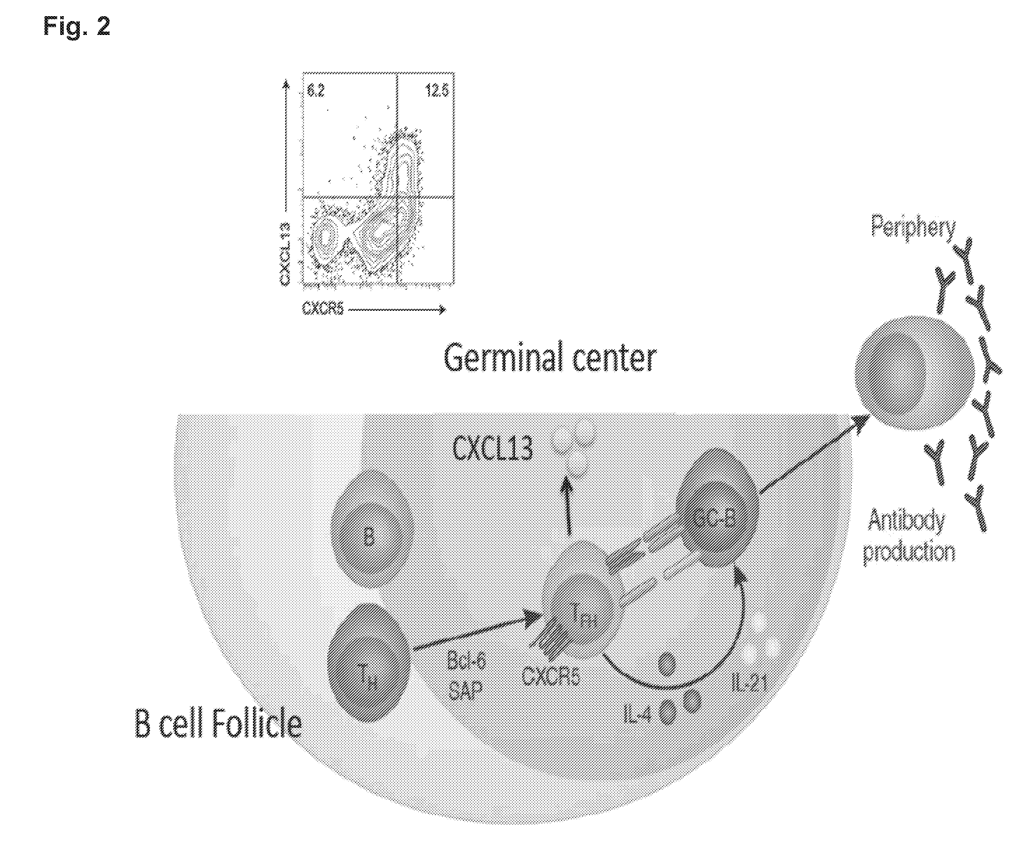

GC Tfh cells are a major producer of CXCL13 in secondary lymphoid tissue such as tonsil (19, 20, 33). It was found that GC Tfh cells were uniquely able to produce CXCL13 when analyzed by intracellular FACS analysis (FIG. 2A). Other cell types have been reported to have the ability to produce CXCL13 under various inflammatory conditions (32) (34) (35). Therefore, it was also examined all other cells present in the tonsil tissue preparations. No other cell type showed CXCL13 production (FIG. 2B). Similar results were obtained from mononuclear cell preparations isolated from human spleen and lymph node tissues, showing CXCL13 expression to be restricted to GC Tfh cells (FIG. 2C and data not shown).

In an additional cohort of HIV+ and HIV− individuals at Massachusetts General Hospital, the present inventor was able to obtain lymph node biopsies allowing direct comparison of plasma CXCL13 to GC activity in human lymphoid tissue. GC Tfh cells (CXCR5hi and PD-1hi) were identified (FIG. 2D) and quantified. In 14 matched plasma and lymph node samples, plasma CXCL13 levels positively correlated with GC Tfh cells in the lymph node (r=0.7484, p=0.003) (FIG. 2E). The strong correlation observed between plasma CXCL13 and lymph node GC Tfh cells within a relatively small human donor set, together with the ability of GC Tfh cells to produce CXCL13 suggests that GC Tfh cell production of CXCL13 is the direct cause of elevated plasma CXCL13 and that CXCL13 could function as biomarker of GC activity.

Plasma CXCL13 is Correlated with Lymphoid Tissue Germinal Centers in Mice after Immunization

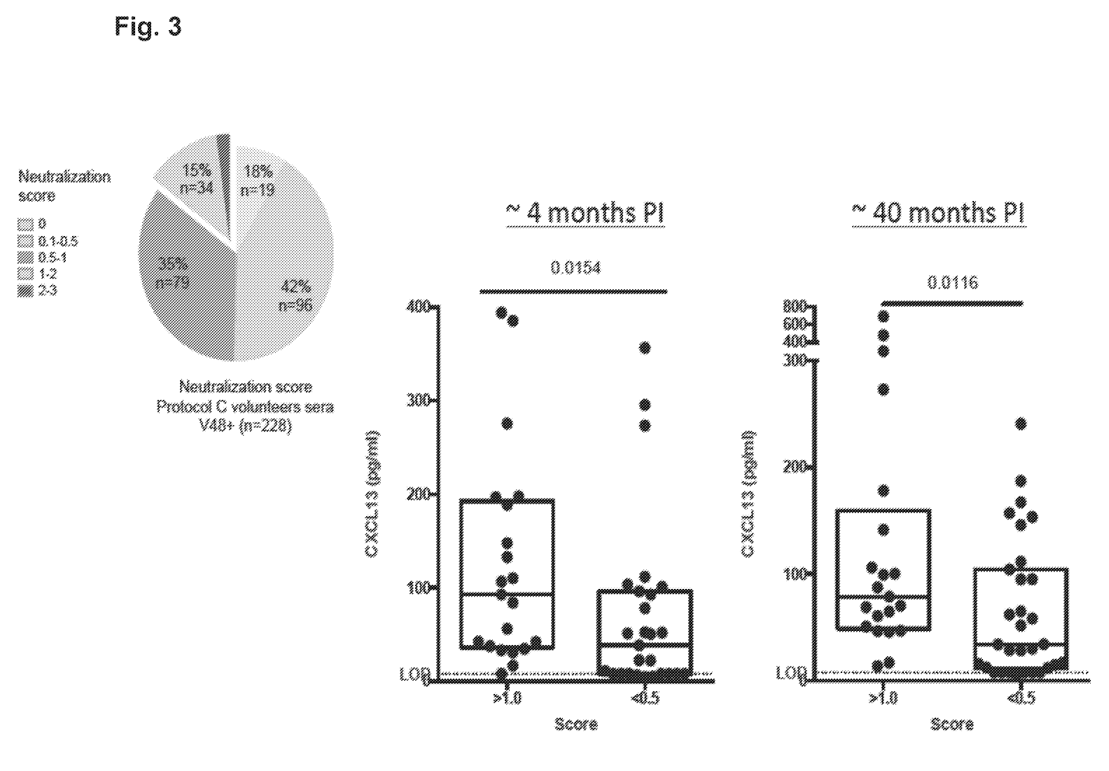

Next, the relationship between plasma CXCL13 and GC activity after protein immunization in animal vaccination models was examined, since leading HIV vaccine candidates aimed at inducing broadly neutralizing antibodies involve the use of recombinant proteins (8, 36-38). In addition, by examining plasma CXCL13 after protein immunization rather than chronic HIV viral infection, any effects of viral load and chronic immune stimulation on plasma CXCL13 can be eliminated. The study was initiated in mice. Mice were immunized with alum plus haptenated ovalbumin (Alum+NP-OVA) to measure plasma CXCL13 after a protein immunization. Half of the immunized mice received OT-II TCR transgenic CD4 T cells to enhance the antigen specific and GC Tfh CD4 T cell response. Seven days after immunization, plasma CXCL13 was increased (FIG. 3A) in comparison to unimmunized mice. Plasma CXCL13 was also increased seven days after acute infection with LCMV Armstrong or a smallpox vaccine stain of vaccinia virus (FIG. 3A). In alum+NP-OVA immunized mice, GC Tfh were identified (CXCR5hi PD-1hi, FIG. 3B). Plasma CXCL13 correlated with the percentage of GC Tfh cells induced in the draining lymph node (FIG. 3C, r=0.8222, p=0.0018).

In a larger kinetic study of mice that did not receive transgenic CD4 T cells, Keyhole limpet hemocyanin plus Alum (KLH+alum) immunized mice had higher plasma concentrations of CXCL13 after immunization in comparison to the pre-immunization time points in the same mice; though with slower kinetics peaking at 2 weeks post immunization (FIG. 3D). This group of mice was then re-immunized with KLH+alum, 50 days after the primary immunization. Plasma CXCL13 concentrations were increased at both 10 and 18 days post boost (FIG. 3E) and again correlated with GC Tfh cells in the draining lymph node (FIG. 3F; r=0.6909, p=0.0226). Even given these encouraging results, mice may not be the best animal model system for analyzing CXCL13, as mouse GC Tfh cells are only capable of producing modest amounts of CXCL13, while human GC Tfh are proficient producers of CXCL13 (39). However, the mouse studies here do support the idea that plasma CXCL13 can be used as a biomarker of GC activity in protein immunization studies.

Plasma CXCL13 is Correlated with Lymphoid Tissue Germinal Centers in Macaques after Immunization

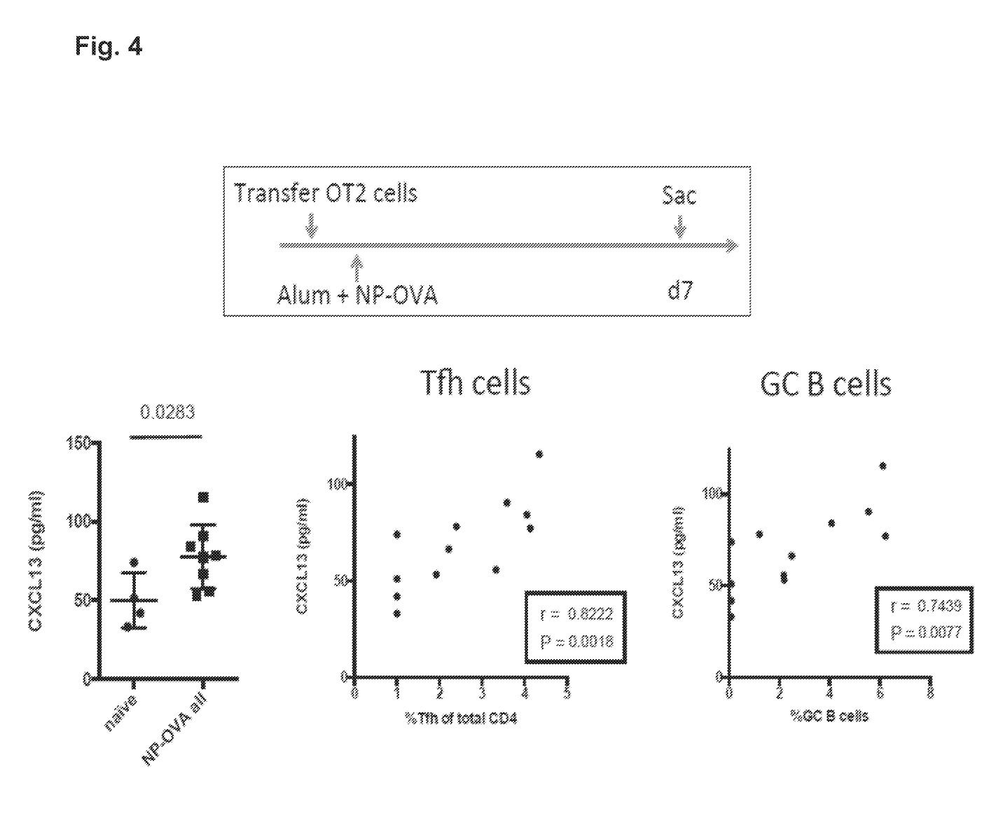

Non-human primates are considered the best animal model for pre-clinical vaccination studies. The present inventor considered that CXCL13 expression by GC Tfh in non-human primates might be more similar to that in humans. Therefore the relationship between plasma CXCL13 and GC activity in rhesus macaques after protein vaccination was examined. The previous identification of an anti-human CXCR5 antibody (clone MU5UBEE) reactive to rhesus macaque CXCR5 (used in (40) and (41)) allowed detection of a CXCR5hi PD1hi GC Tfh population in macaque lymphoid tissue (FIG. 4A). The GC Tfh population in macaques highly expresses Bcl-6, ICOS, and CD200 (FIG. 4A) analogous to CXCR5hi PD-1hi GC Tfh cells in humans. Seven days after a protein+adjuvant immunization, GC Tfh and GC B cells responses were found in the draining lymph node and not a non-draining lymph node (FIG. 4B). The plasma CXCL13 in these same animals was examined at the same day 7-post immunization time point and again found a positive correlation between plasma CXCL13 and GC Tfh cells in the draining lymph node (FIG. 4C; r=0.7263, p=0.0129). The correlation between plasma CXCL13 and GC Tfh in immunized rhesus macaques was confirmed in an additional study of 12 animals (Havenar-Daughton C, Kasturi S, Pulendran B, and Crotty S, unpublished). In summary, plasma CXCL13 levels in both mice and rhesus macaques strongly correlates with GC Tfh cell frequencies in the draining lymph node after immunization.

Plasma CXCL13 is Increased after Immunization in Humans

Plasma CXCL13 can be detected in human plasma both under normal physiological conditions and during infections, autoimmune diseases, and cancer. However, little is known about the dynamics of plasma CXCL13 in humans after vaccination. Plasma CXCL13 levels after vaccination in humans were investigated herein to determine if plasma CXCL13 is both elevated after immunization in animal models and correlated with GC activity. If measurable in humans after immunization, plasma CXCL13 could be a useful parameter to monitor lymph node GC activity during clinical vaccine trials.

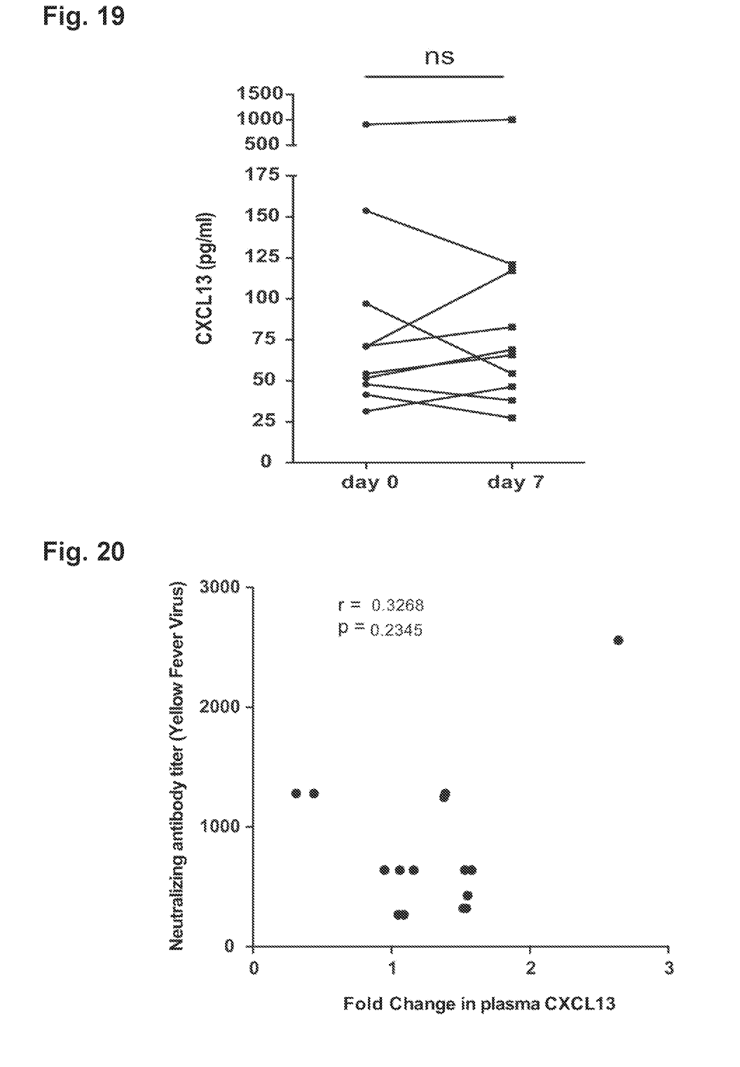

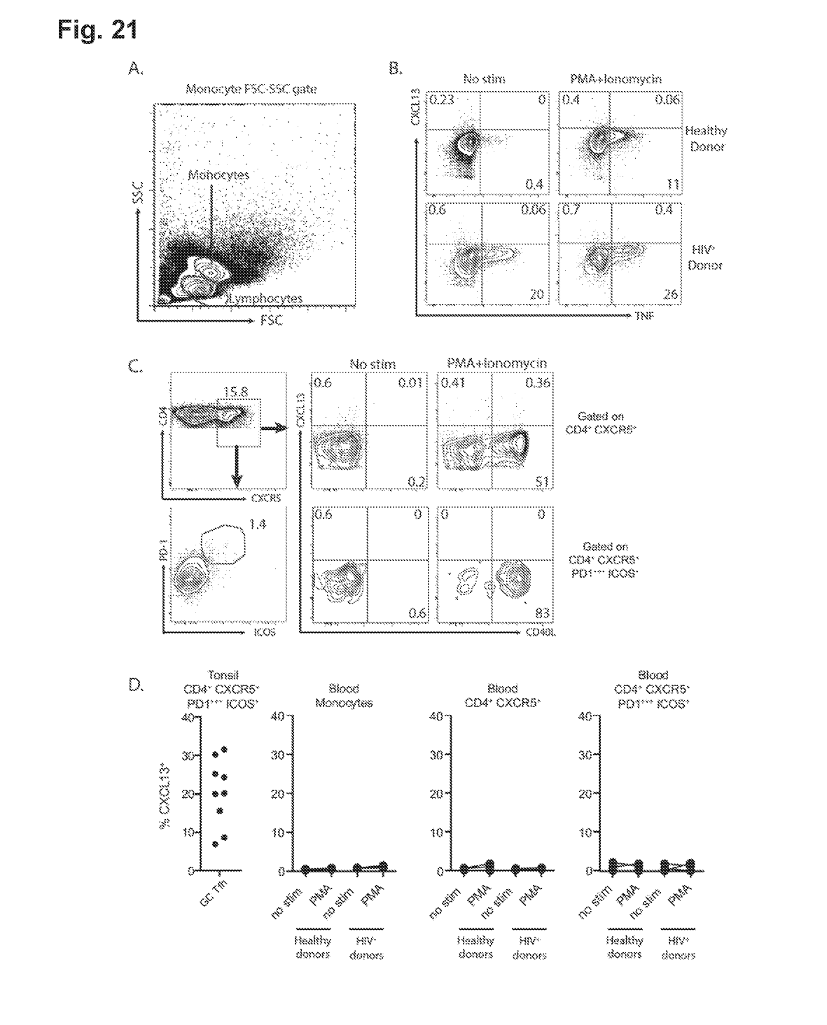

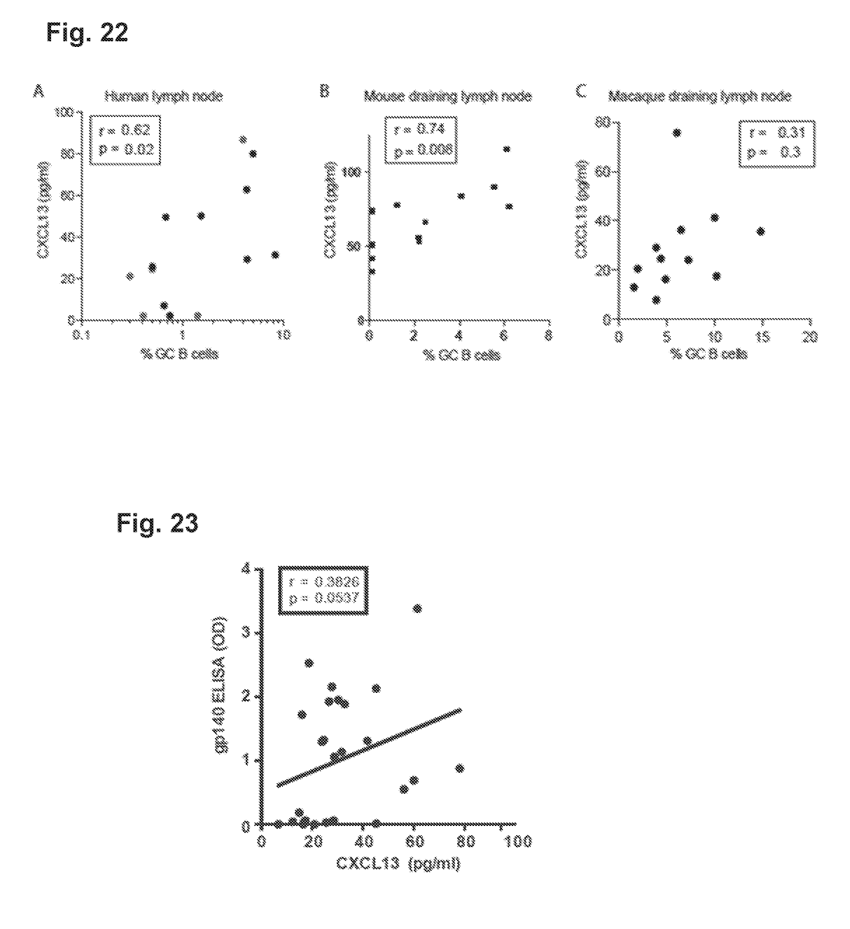

Plasma CXCL13 were initially examined in a small cohort of influenza vaccine recipients. Mixed plasma CXCL13 responses were found after influenza immunization that did not show overall statistically significance change at the cohort level (FIG. 19). The lack of a clear increase in plasma CXCL13 could be due to a low overall response generated to the immunization because of pre-existing antibody titers already present. Indeed, only 2 of the 10 individuals tested had more than 2 fold increases in neutralizing antibody titers against more than 1 influenza strain after vaccination (data not shown). Therefore our analysis focused on immunizations that generated novel immune responses. Two groups of immunized humans were examined. The first cohort was immunized with an FDA-approved yellow fever vaccine. The second group was made up of study participants in an HIV Vaccine Trials Network protocol testing a candidate HIV vaccine regimen.

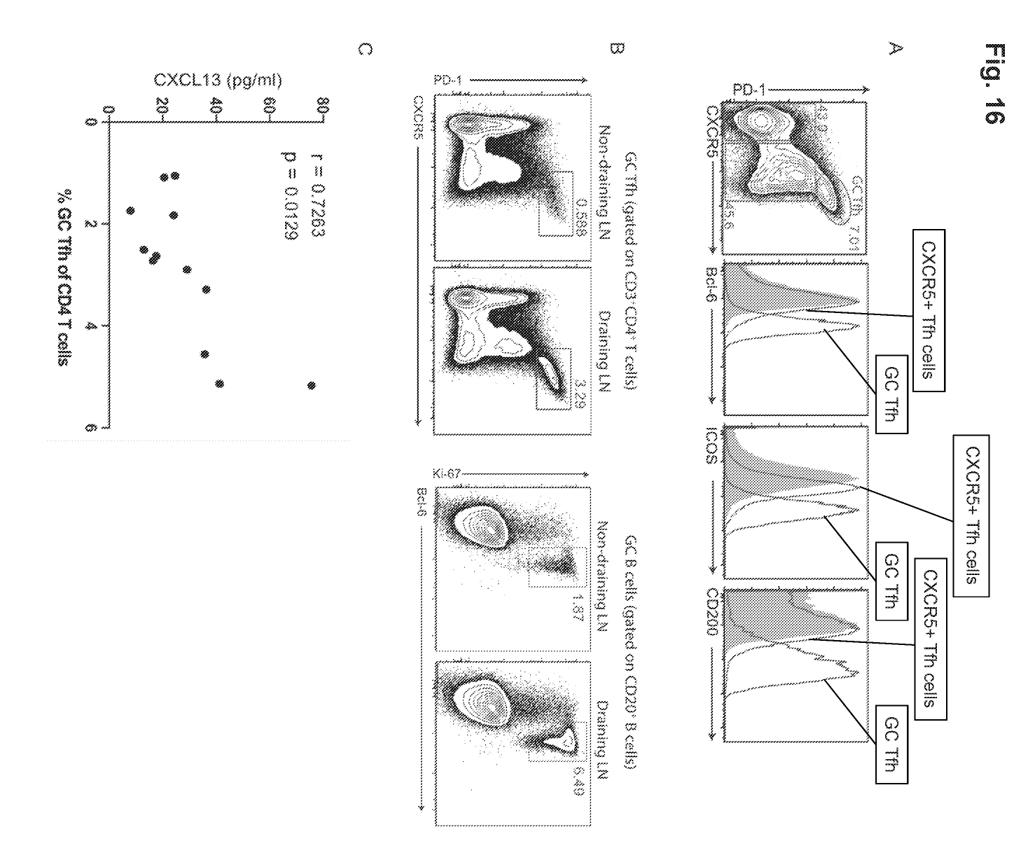

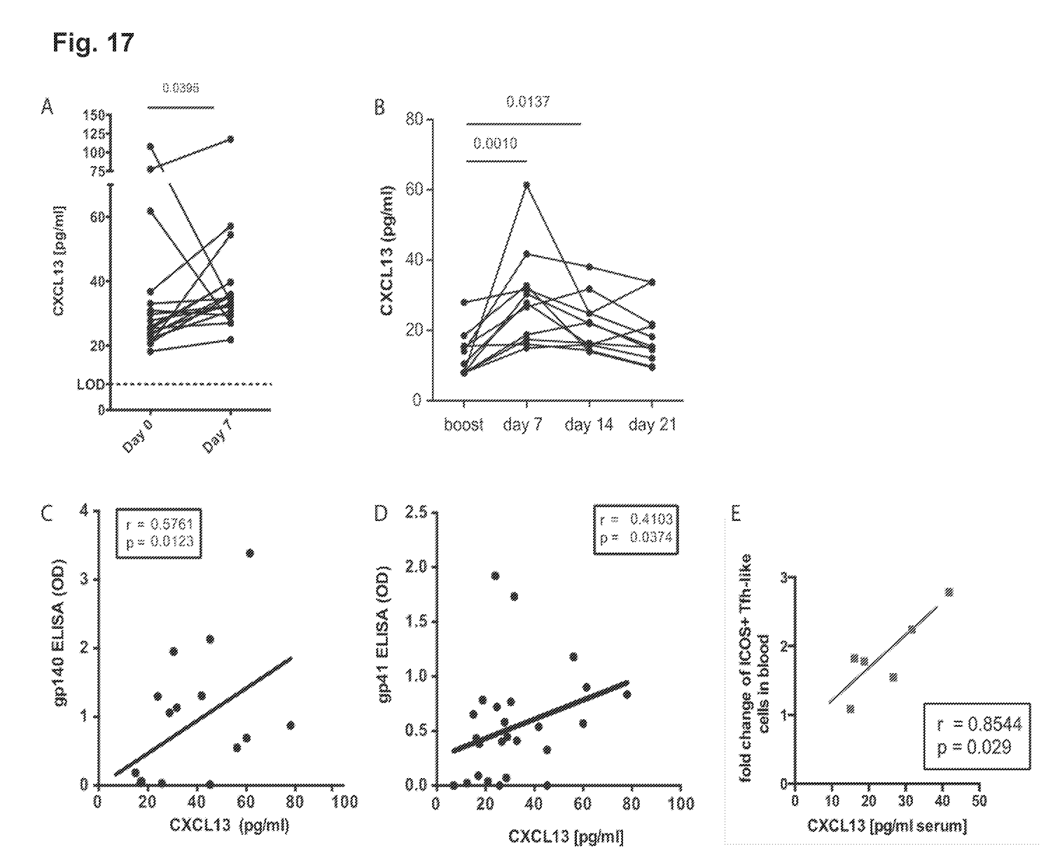

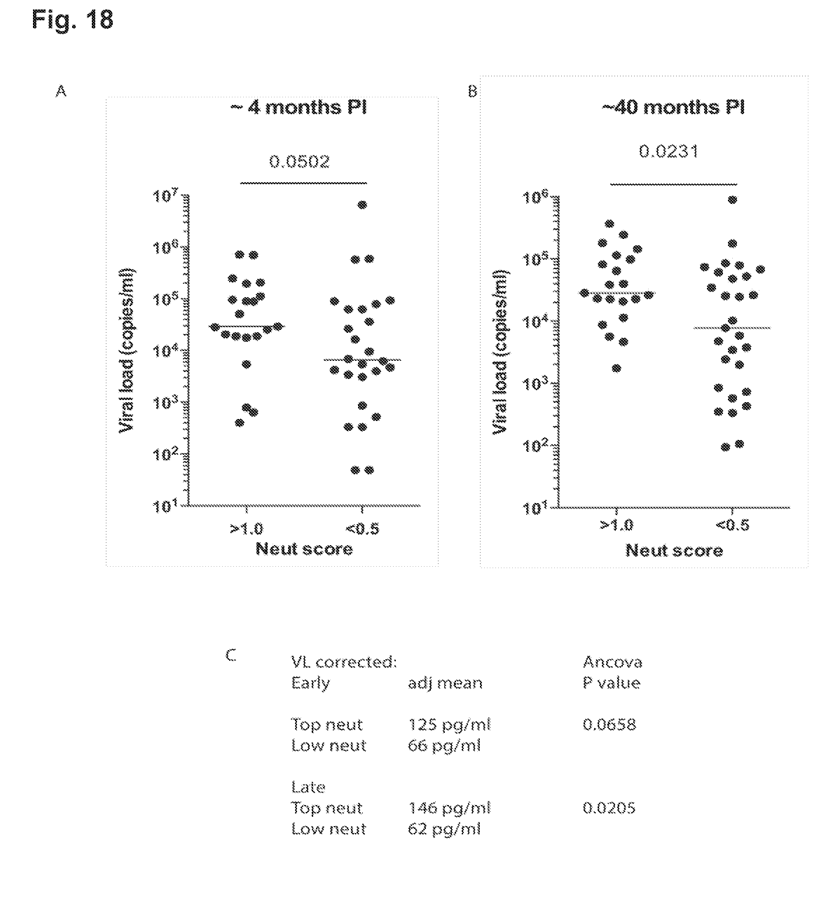

Pre- and post-vaccination plasma samples obtained from 17 yellow fever vaccine recipients were tested. Seven days after immunization, modest but statistically significantly increases in plasma CXCL13 were observed (FIG. 17A; p=0.0395). Plasma CXCL13 was 30% higher than pre-vaccinations levels in 9 of 17 individuals. A non-significant positive trend was observed between the fold increase in CXCL13 after vaccination and the yellow fever neutralizing antibody titer measured 4 weeks after immunization (FIG. 20).

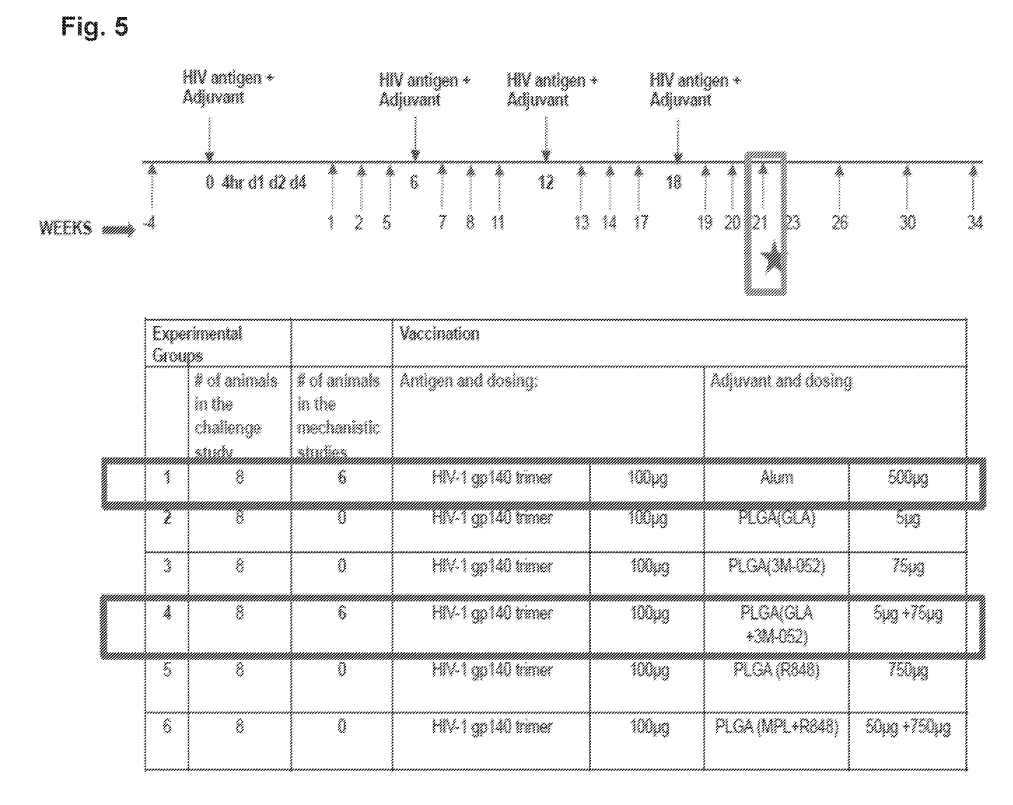

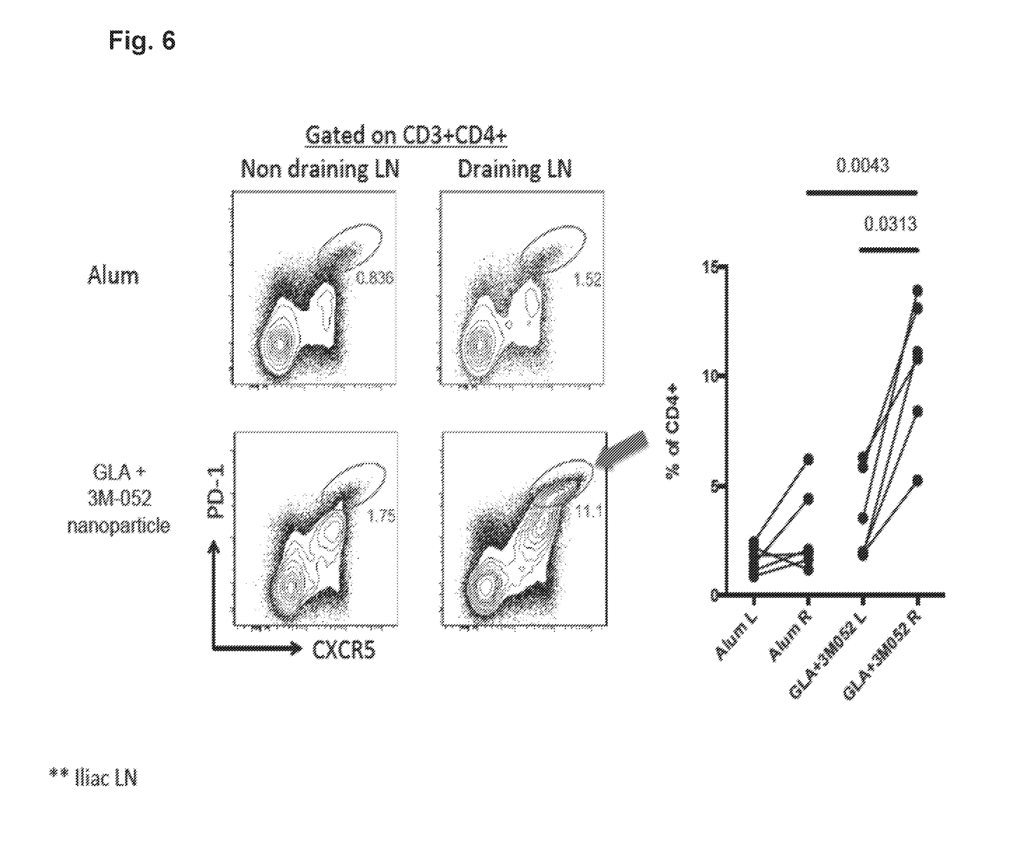

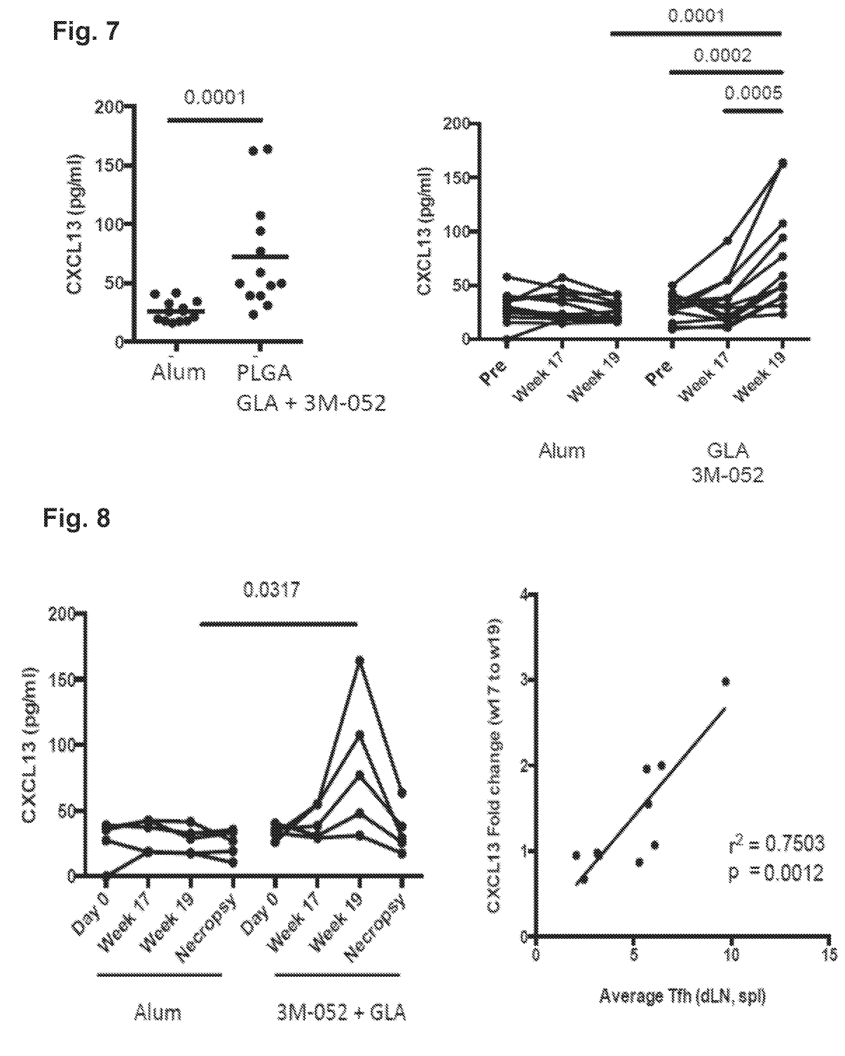

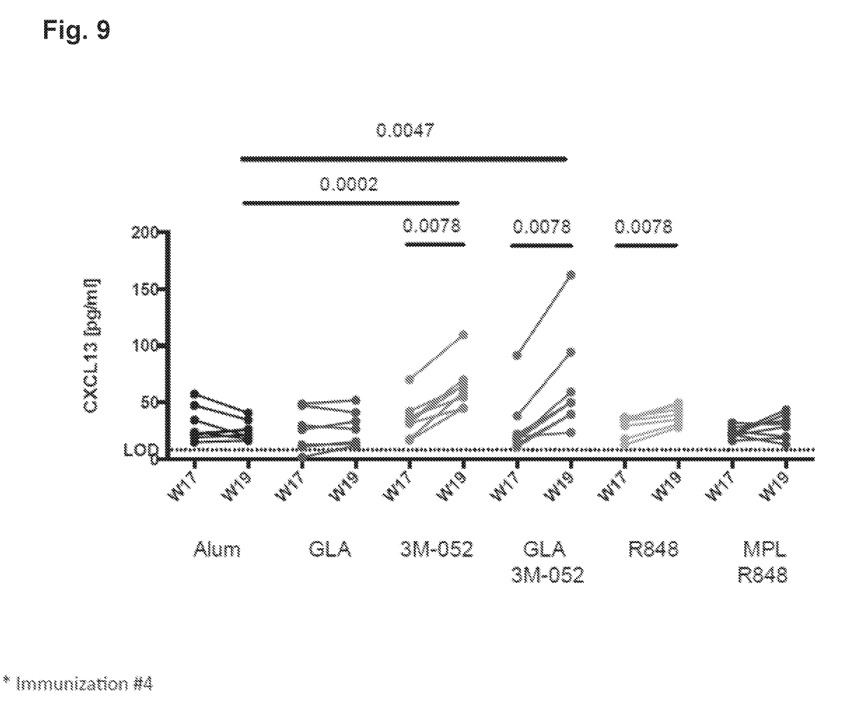

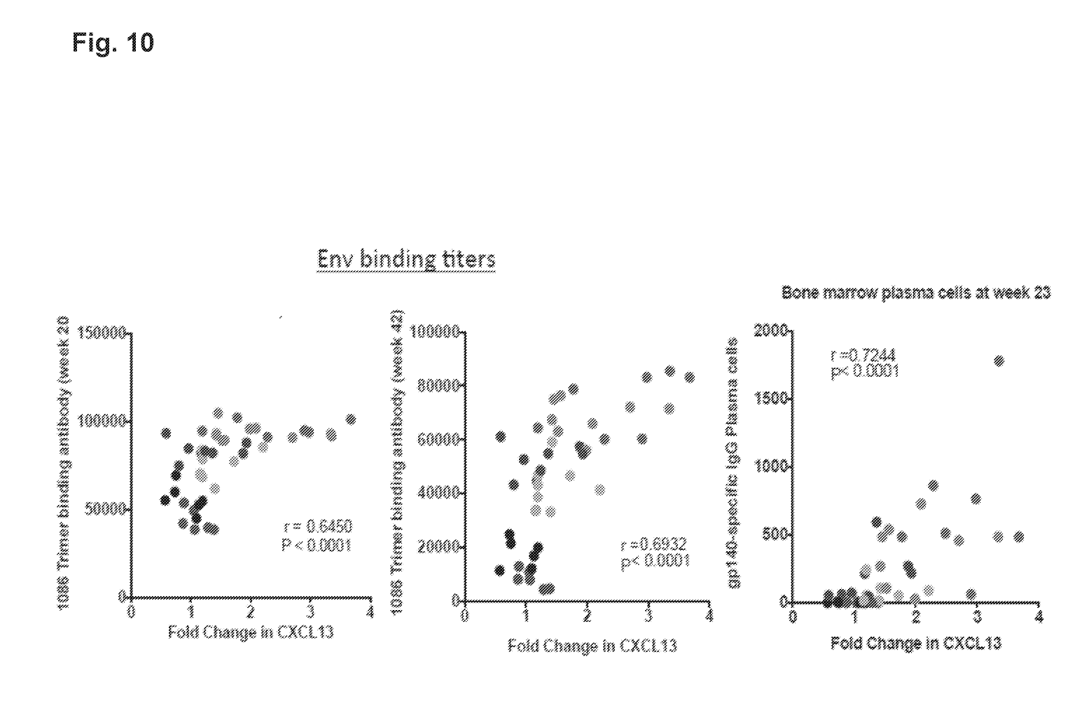

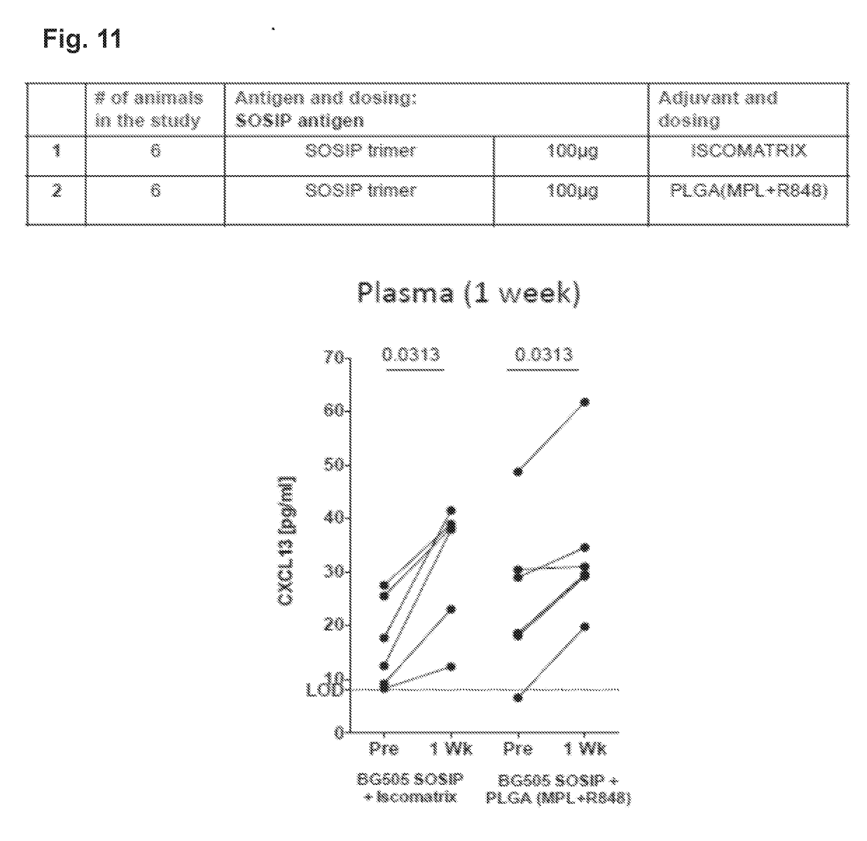

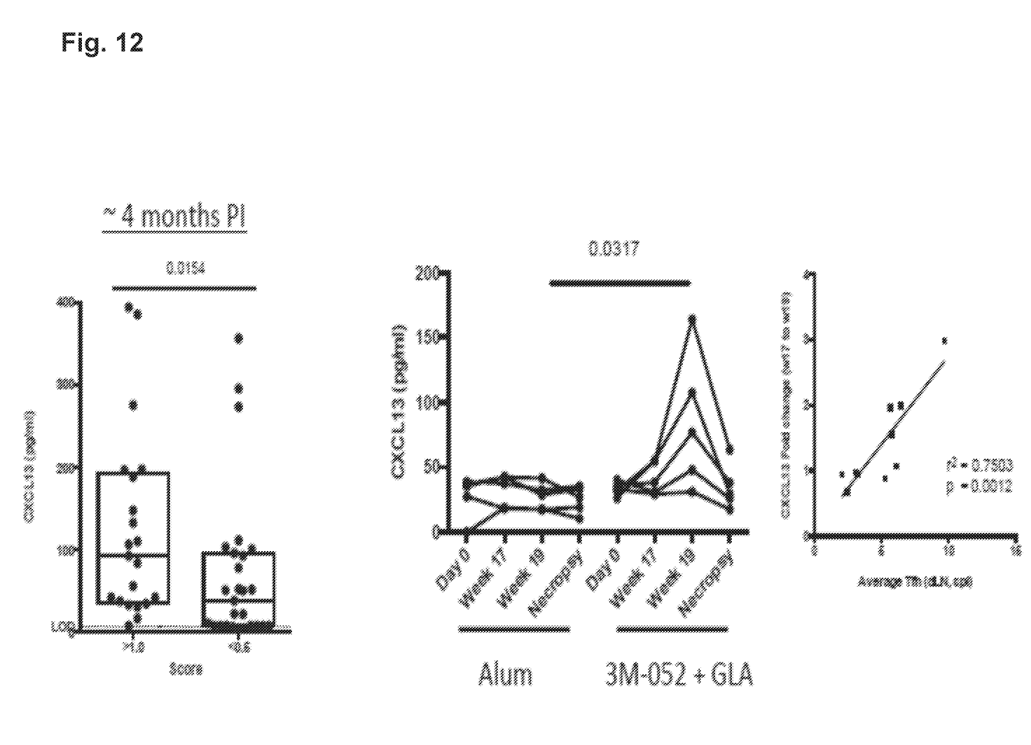

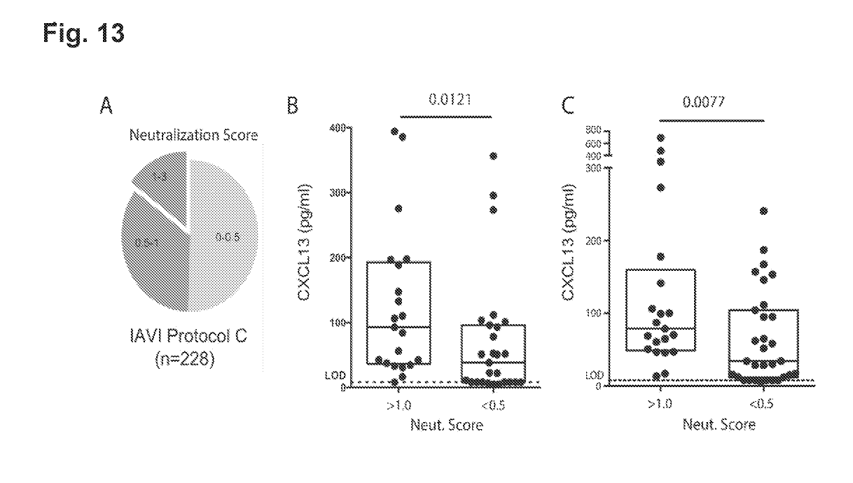

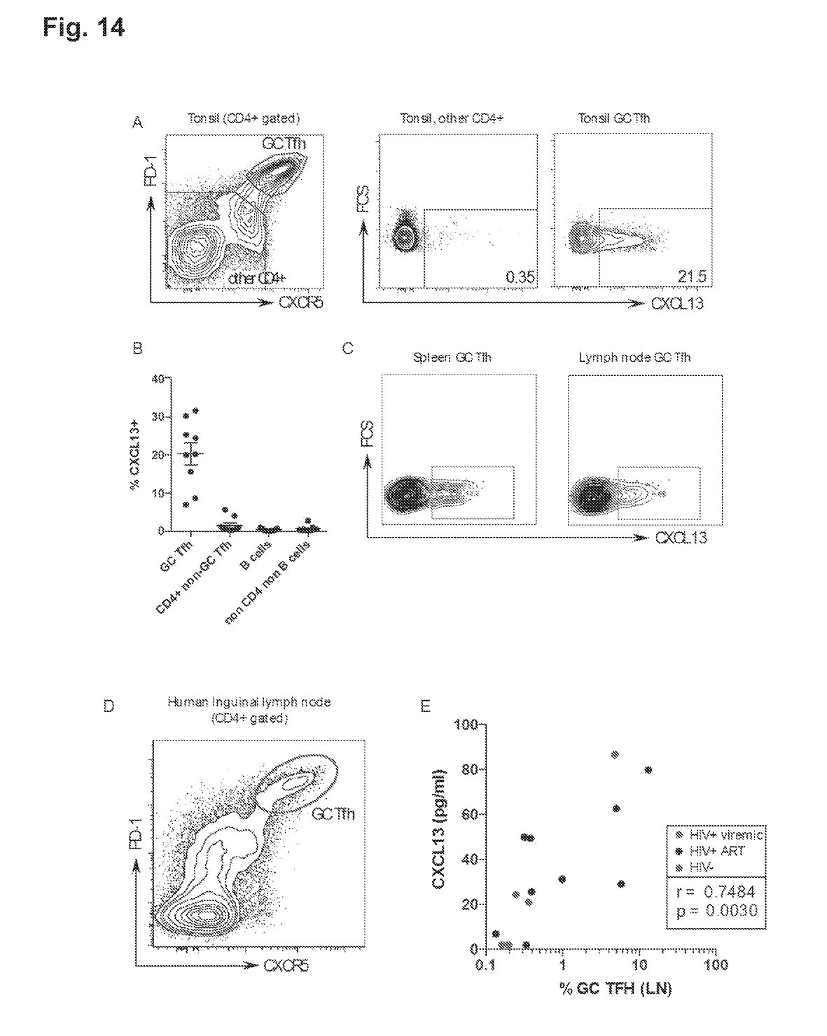

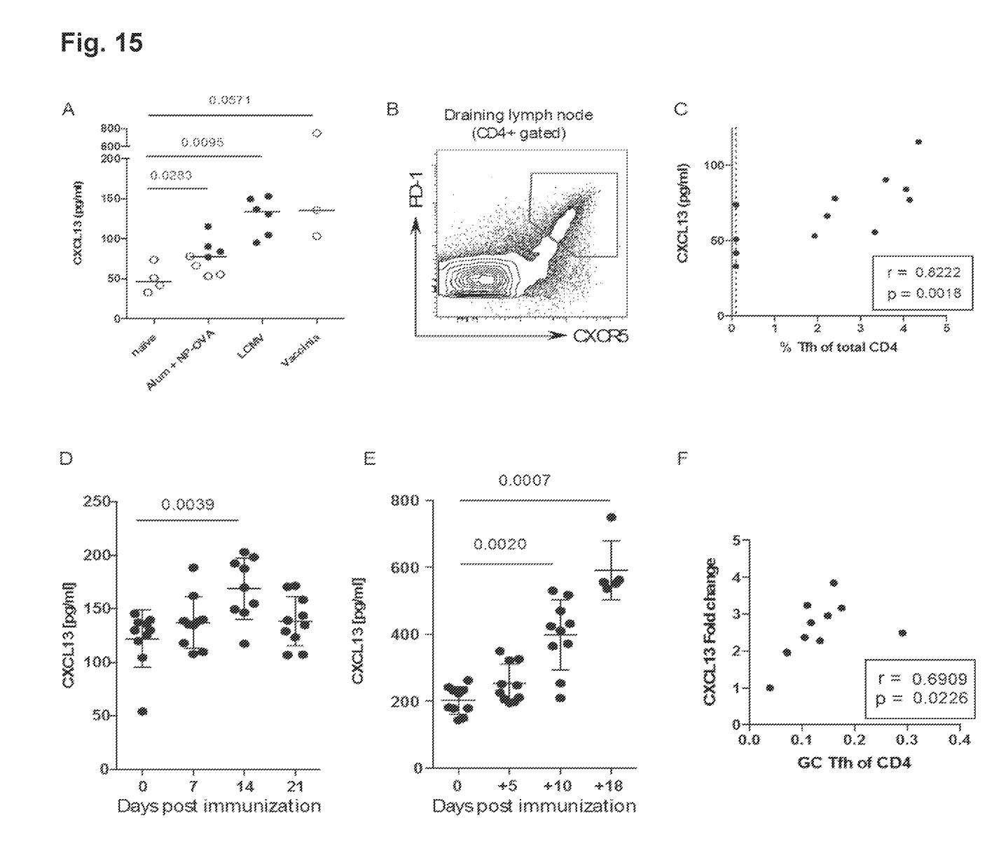

To confirm the results found after yellow fever vaccination, the kinetics of plasma CXCL13 levels in 11 vaccinated individuals in HVTN 068 were assessed (43). Pre-boost (Ad5/HIV vector) and day 7, 14, and 28 post-boost samples were available for analysis. Plasma CXCL13 was significantly increased over pre-boost levels at both day 7 (p=0.001) and day 14 (p=0.0137) (FIG. 17B). For each individual donor, peak plasma CXCL13 was detected at either the 7 or 14-day time point. Seven days after vaccination, plasma CXCL13 was 50% higher than the pre-vaccination time point in 9 of 11 individuals. In a larger set of samples available at only the 7-day time point, plasma CXCL13 positively correlated with the vaccine-specific gp140 Env IgG antibody response (FIG. 5C; r=0.5761, p=0.0123) and the gp41 Env IgG antibody response (FIG. 17D; r=0.4103, p=0.0374) at 28 days after immunization. In a few individuals for which cryopreserved PBMC were available, the frequency of small, activated ICOS+PD1+CXCR5+CD4 T cells after vaccination were evaluated. ICOS+PD1+CXCR5+CD4 T cells are found in the blood of individuals with ongoing immune responses and have a similar, but not identical phenotype to GC Tfh cells (29, 44, 45). Among 6 individuals analyzed, day 7 plasma CXCL13 correlated with the increase in activated ICOS+PD1+CXCR5+CD4 T cells found in the blood 7 days after immunization (FIG. 17E). Here, are shown examples of detectable increases in plasma CXCL13 at 7-14 days post immunization in vaccinated humans and its correlation with the antibody response to vaccination and the ICOS+PD1+CXCR5+CD4 T cell population, making it a potentially useful biomarker to monitor during human vaccine trials.

### Discussion

The GC response is a critical immune mechanism by which antibody affinity, memory B cell, and plasma cell development occurs. If broadly neutralizing antibodies against HIV are to be generated by vaccination, the GC response will play a central role. Here, there is provided a novel means to monitor GC activity in lymphoid tissues using a plasma biomarker. Plasma CXCL13 positively correlates with the lymph node GC response in mice, macaques, and humans. In each case, the relationship between plasma CXCL13 and GC Tfh cells is strong as the correlation coefficient is high. Increases in plasma CXCL13 were found in a number of different immune activating conditions: alum or TLR ligand adjuvants plus recombinant protein immunizations, adenovirus vector vaccine and attenuated yellow fever vaccine administration, and HIV infection. Based on the strong correlation of GC Tfh and plasma CXCL13, and the significant measurable change in plasma CXCL13 in two human vaccine cohorts, monitoring plasma CXCL13 is useful in human and NHP vaccine trials where direct analysis of lymphoid tissue is either not possible or undesirable for fear of disturbing the ongoing immune response.

A strong correlation between CXCL13 and lymphoid tissue resident GC Tfh cells was shown. With the additional observation that GC Tfh cells are robust producers of CXCL13, a direct relationship between GC Tfh and plasma CXCL13 is suggested. Other cell types in lymph tissue that produce CXCL13 were not identified by intracellular cytokine analysis, although FDC and some dendritic cell subsets are likely lost in tissue processing. During the GC response, GC Tfh develop and expand in number (i.e., amount) potentially explaining the increased levels of CXCL13 found in the plasma. GC Tfh cells are not likely to be the only source of increased plasma CXCL13 after immunization as FDC are known producers of CXCL13 and also expand in number during the GC response. However, a histological study suggests that much of the CXCL13 observed in the tonsil GC co-stains with PD-1, a marker of GC Tfh rather than CD21, a marker of FDC (33). Other cellular sources of CXCL13 have also been reported in various disease models and could contribute to plasma CXCL13 concentrations. Monocytes (32), macrophages (34), and dendritic cells (35) have been reported to express CXCL13 in different inflammatory settings. The recent finding by Cohen, et al that monocytes can produce CXCL13 in response to IFN-I may be relevant in HIV infection and immunizations with viral vectors or adjuvants containing TLR ligands. However, IFN-I responses are normally very early and short in duration, peaking at 24-48 hours after immunization (46, 47). Therefore, monocyte generated CXCL13 may not greatly impact plasma CXCL13 measured at the later time points (day 7-14) in our immunization studies. Instead, these later time points are contemporaneous with GC activity. While the cellular source of plasma CXCL13 after immunization is not definitively identified here, GC Tfh are key players in the GC reaction, producers of CXCL13, and their quantities positively correlate with plasma CXCL13.

CXCL13 is detectable in human plasma and is elevated 7 to 14 days post immunization. Significant increases of plasma CXCL13 in individuals immunized with either the FDA-approved yellow fever vaccine or an adenovirus vector based candidate HIV vaccine were detected. However, mixed plasma CXCL13 responses after influenza immunization that did not show overall statistical significance at the cohort level were found. The lack of a clear increase in plasma CXCL13 after influenza immunization could be due to a lower overall response generated by immunization due to pre-existing antibody titers. This issue was avoided in the adenovirus vector-based candidate HIV vaccine trial, as vaccine recipients were naive to HIV-1 envelope antigens at study enrollment and pre-screened for low pre-existing adenovirus antibody titers (43). Pre-existing antibodies may lead to fewer germinal centers being generated and therefore lack of significant changes in plasma CXCL13. It is interesting that plasma CXCL13 was significantly elevated after the robust yellow fever vaccination, but less clearly elevated after the less clinically robust influenza immunizations. In any case, plasma CXCL13 is a useful parameter to measure in human and NHP vaccine trials.

Elevated plasma CXCL13 was associated with the generation of broadly neutralizing antibodies in the large ART-naïve HIV+ IAVI Protocol C cohort. It is unclear why only a few HIV-infected individuals are able to generate bnAb. Virologic factors clearly play an important role in the generation HIV bnAb (30, 48, 49). In addition, a memory population of PD1+CXCR3− CXCR5+CD4+ T follicular helpers cells circulating in the blood were shown to be associated with the ability to generate HIV bnAb (29). Although plasma CXCL13 and the PD1loCXCR3− CXCR5+CD4+ memory Tfh population did not correlate in the IAVI Protocol C cohort (data not shown), both are associated with HIV bnAb generation. Independently, CXCL13 has been reported to be correlated with increased neutralizing antibody breadth in a small cohort (n=15) of HIV+ individuals (50). In addition, plasma CXCL13 is correlated with GC Tfh cells in human LN in a cohort of HIV+ and HIV-individuals. An intriguing implication of this strong correlation is the suggestion that biopsy of a single lymph node is fairly representative of global GC activity in an HIV+ individual. Together, the association of both CXCL13, a biomarker for GC activity, and memory Tfh cells with the development of bnAb against HIV suggests that these individuals have immune systems capable of continuously productive GC responses, leading to higher levels of SHM, and resulting in generation of HIV bnAb.

Data presented herein showing the relationship between plasma CXCL13 and GC activity may partially explain an association between CXCL13 and autoimmune disease by pointing to a role for antibody and GCs. However, the CXCL13 signal found in autoimmune individuals is likely complex. In addition, it is important to note that analysis of plasma CXCL13 is not an antigen or disease specific readout. Plasma CXCL13 reports total GC activity. In some embodiments, for example after immunization, plasma CXCL13 is analyzed together with antigen-specific antibody and T cell responses, and/or the appearance of an activated Tfh-like population of circulating ICOS+PD1+CXCR5+CD4 T cells to best monitor the ongoing immune response.

Here, it is shown that CXCL13 acts as a plasma biomarker for GC activity in generally inaccessible lymphoid tissue. Plasma CXCL13 correlates with GC activity after immunization in animal models and in HIV+ humans. Furthermore, plasma CXCL13 is associated with generation of HIV bnAbs and elevated after immunization in humans. Together, these findings support the use of CXCL13 as a plasma biomarker of germinal center activity in human vaccine trials and other clinical settings.

### Material and Methods

### IAVI Protocol C

The IAVI Protocol C cohort has been described elsewhere (29). A more extensive explanation of the cohort and testing for neutralizing antibodies will be published elsewhere (Landais E. and Poignard P, unpublished data). A HIV-neutralization score for each plasma sample was determined as in Simek et al. (58) to account for both breadth and potency. Individuals reaching a score of >1 were labeled as top neutralizers, while individuals with scores <0.5 were labeled as low neutralizers. Plasma samples tested for CXCL13 were from the earliest available time point (˜4 months, range 0-9 months post-infection) and at the time of bnAb development (˜40 months, range 24-54 months post infection by which time most Top neutralizers had developed neutralizing breadth). All donors were ART-free and had CD4 counts >200 cells/ml at each time point tested. The study was approved by La Jolla Institute and Scripps Research Institute Internal Review Boards. Written consent was obtained from all study participants before enrollment in the study.

### Human Lymph Node, Spleen, and Tonsil

Inguinal lymph nodes from HIV infected persons and HIV-negative healthy volunteers were obtained by excisional surgical biopsy under local anesthesia at Massachusetts General Hospital (MGH) in Boston and processed as in Lindqvist, et al (59). The study was approved by the Partners Human Research Committee and written consent was obtained from all study participants before enrollment in the study. Non-identifiable excess spleen and lymph node tissue was obtained at Massachusetts General Hospital, Boston under approval by the Partners Human Research Committee. These surgeries were performed for medical or surgical indications not related to hematologic or autoimmune diseases. Discarded human tonsil tissue was obtained from the Rady Children's Hospital, San Diego. Informed consent was obtained from all donors. Tissues were cut into small fragments and mechanically disrupted using a 70-um cell strainer or wire mesh and a syringe plunger. Following disruption, spleen cells were treated with RBC lysis buffer (Qiagen) and tonsil mononuclear cells were isolated on a ficoll gradient (19). Cells were then washed and used for experiments or cryopreserved for later use.

### Human Vaccine Cohorts

Plasma from influenza vaccine recipients from the Stanford-Lucile Packard Children's Hospital Vaccine Program was analyzed for CXCL13 pre- and 1 week post immunization (42, 60). The original study was approved by the Institutional Review Board of the Research Compliance Office at Stanford University. Participants were immunized with a single dose of TIV Fluzone (Sanofi Pasteur). For yellow fever vaccine recipients plasma was analyzed for CXCL13 pre- and 1 week post immunization. Detailed clinical trial information has been published elsewhere (61). Participants were immunized with the Food and Drug Administration (FDA)-approved 17D YF vaccine. The original study was approved by the Institutional Review Boards at Emory University and the Centers for Disease Control (CDC). Plasma samples from HIV-uninfected candidate vaccine recipients from the HVTN 068 trial were analyzed for CXCL13 levels in plasma collected pre-, 7 day, 14 day, and 28 days after a booster immunization (adenovirus-5 vector with HIV-1 gene inserts; [Ad5/HIV]) given at the 6 month time point. Primary immunizations were either Ad5/HIV at month 0 or DNA/HIV at months 0 and 1. Some participants received placebo. Detailed clinical trial information and results have been published elsewhere (43). The original study was approved by the Institutional Review Board at the HIV Vaccine Trials Network (HVTN). Informed consent was obtained from all subjects.

### Mouse Immunization

C57BL/6 (B6) mice were purchased from the Jackson Laboratory. For alum plus 4-Hydroxy-3-nitrophenyl acetyl haptenated ovalbumin (NP-OVA; Sigma) immunized mice, bug NP-OVA was mixed 1:1 with 10% alum (aluminum potassium sulfate dodecahydrate (Sigma)) in PBS. In some mice, 2×105 OVA-specific OT-II TCR transgenic CD4 T cells were transferred 3.5 days prior to immunization. For keyhole limpet hemocyanin plus Alum (KLH+Alum) immunized mice, bug KLH was mixed 1:1 with 10% alum in PBS. For LCMV immunization experiments, 2×105 NIP CD4+ TCR transgenic cells (62) were transferred prior to immunization with 2×106 PFU of LCMV Armstrong by intraperitoneal injection. For Vaccinia virus immunization experiments, B6 mice were intraperitoneally injected with 6×10⁵ PFU. All animal procedures were performed in accordance with approved animal protocols at La Jolla Institute for Allergy and Immunology.

### Macaque Immunization Study

Plasma samples were analyzed from Indian rhesus macaques before and 1 week after either the first or second booster immunization. Macaques were immunized subcutaneously with gp140 (SIVmac239) Env and p55 Gag protein mixed with either alum (Aluminum hydroxide; Alhydrogel 2% adjuvant, Invivogen) or MPL+R848 encapsulated PLGA nanoparticles (63). A full description of the vaccine trial methods and results will be published elsewhere (Kasturi S. and Pulendran B.) All animal procedures were performed in accordance with guidelines established by the Emory School of Medicine Institutional Animal Care and Use Committee Guidelines.

### CXCL13 ELISA

Instructions were followed as per the Human CXCL13 Quantikine ELISA kit from R&D Systems. In short, plasma samples were thawed and incubated on the provided 96-well plate with assay diluent at room temperature for 2 hours. The plate was washed; a detection antibody reagent was added and incubated again for 2 hours at room temperature. After washing, a substrate solution was added to the plate and allowed to react for 30 minutes at room temperature away from light. The reaction was stopped with the provided stop solution and absorbance was read at 450 nm. All samples were tested in duplicate. The human CXCL13 Quantikine kit from R&D Systems was found to have superior specificity and reproducibility in comparison to the CXCL13 DuoSet (R&D Systems) or human CXCL13 ELISA kit from Sigma-Aldrich (data not shown). The Human CXCL13 Quantikine ELISA kit was also used for quantification of CXCL13 in macaque plasma. Anti-human CXCL13 antibody reagents from R&D Systems have previously been used to detect macaque CXCL13 (64). The Mouse CXCL13 DuoSet (R&D Systems) was used for quantification of CXCL13 in mouse serum as described in the instructions and similar to the human CXCL13 ELISA described above.

### Flow Cytometry Analysis of GC Tfh in Mice, Macaques, and Humans

GC Tfh cells were characterized as previously described in mouse lymph node (65), human tonsil (19, 29), or human lymph node (59). GC Tfh cells in macaque lymph node were identified with a staining panel consisting of antibodies against CXCR5 (MU5UBEE, eBioscience), ICOS (C398.4A, BioLegend), CD200 (OX104, eBioscience), PD-1 (EH12.1, BD Biosciences), Bcl-6 (K112-91, BD Biosciences), CD4 (OKT4, BioLegend), CD3 (SP34-2, BD Biosciences), CD20 (2H7, BioLegend), and Fixable Viability Dye (eBioscience). Cells were acquired on BD Fortessa analyzer. Previously cryopreserved peripheral blood mononuclear cells (PBMC) from HVTN 068 subjects were stained and acquired on a BD LSRII flow cytometer as described (Heit et al, submitted).

### CXCL13 Staining in Human Cells

Cells were thawed and rested in R10 media at 37° C. overnight. Cells were then incubated for 4 hours at 37° C., in the presence of Brefedin A, without any stimulation. Cells were then stained for CXCR5, CD19, CD4, PD1, and a live/dead cell marker for 1 hour at 4° C., washed, fixed with 1% formaldehyde (PolySciences, Inc.) for 20 minutes at 4° C., then washed and permeabilized with 0.5% saponin (Sigma) for 20 minutes. Cell were then stained with an anti-CXCL13 antibody (R&D Systems) in 0.5% saponin for one hour at 4° C., washed, and acquired on a BD Fortessa analyzer.

### Statistics

A two-tailed non-parametric Mann-Whitney test was used for evaluating differences among groups. A two-tailed, paired, non-parameteric Wilcoxon test was used to evaluate differences between time points for the same individuals. A two-tailed, non-parametric Spearman correlation test was used for all correlative analysis. Covariance of plasma CXCL13 and viral load was evaluated with the ANCOVA multivariate statistical test (VassarStats). Prism 5.0 (GraphPad) was used for all other data statistical analyses.

### Example 2—References and Notes

1. G. D. Victora, M. C. Nussenzweig, Germinal centers, Annu. Rev. Immunol. 30, 429-457 (2012).
2. L. M. Walker, M. Huber, K. J. Doores, E. Falkowska, R. Pejchal, J.-P. Julien, S.-K. Wang, A. Ramos, P.-Y. Chan-Hui, M. Moyle, J. L. Mitcham, P. W. Hammond, 0. A. Olsen, P. Phung, S. Fling, C.-H. Wong, S. Phogat, T. Wrin, M. D. Simek, P. G. Principal Investigators, W. C. Koff, I. A. Wilson, D. R. Burton, P. Poignard, Broad neutralization coverage of HIV by multiple highly potent antibodies, Nature 477, 466-470 (2011).
3. T. Zhou, J. Zhu, X. Wu, S. Moquin, B. Zhang, P. Acharya, I. S. Georgiev, H. R. Altae-Tran, G.-Y. Chuang, M. G. Joyce, Y. Do Kwon, N. S. Longo, M. K. Louder, T. Luongo, K. McKee, C. A. Schramm, J Skinner, Y. Yang, Z. Yang, Z. Zhang, A. Zheng, M. Bonsignori, B. F. Haynes, J. F. Scheid, M. C. Nussenzweig, M. Simek, D. R. Burton, W. C. Koff, J. C. Mullikin, M. Connors, L. Shapiro, G. J. Nabel, J. R. Mascola, P. D. Kwong, N. C. S. Program4, Multidonor Analysis Reveals Structural Elements, Genetic Determinants, and Maturation Pathway for HIV-1 Neutralization by VRC01-Class Antibodies, Immunity, 1-14 (2013).
4. J. Huang, G. Ofek, L. Laub, M. K. Louder, N. A. Doria-Rose, N. S. Longo, H. Imamichi, R. T. Bailer, B. Chakrabarti, S. K. Sharma, S. M. Alam, T. Wang, Y. Yang, B. Zhang, S. A. Migueles, R. Wyatt, B. F. Haynes, P. D. Kwong, J. R. Mascola, M. Connors, Broad and potent neutralization of HIV-1 by a gp41-specific human antibody, Nature 491, 406-412 (2013).
5. B. F. Haynes, M. A. Moody, M. Alam, M. Bonsignori, L. Verkoczy, G. Ferrari, F. Gao, G. D. Tomaras, H.-X. Liao, G. Kelsoe, Progress in HIV-1 vaccine development, J. Allergy Clin. Immunol. 134, 3-10-quiz 11 (2014).
6. D. Sok, U. Laserson, J. Laserson, Y. Liu, F. Vigneault, J.-P. Julien, B. Briny, A. Ramos, K. F. Saye, K. Le, A. Mahan, S. Wang, M. Kardar, G. Yaari, L. M. Walker, B. B. Simen, E. P. St John, P.-Y. Chan-Hui, K. Swiderek, S. H. Kleinstein, S. H. Kleinstein, G. Alter, M. S. Seaman, A. K. Chakraborty, D. Koller, I. A. Wilson, G. M. Church, D. R. Burton, P. Poignard, The effects of somatic hypermutation on neutralization and binding in the PGT121 family of broadly neutralizing HIV antibodies, PLoS Pathog 9, e1003754 (2013).
7. F. Klein, R. Diskin, J. F. Scheid, C. Gaebler, H. Mouquet, I. S. Georgiev, M. Pancera, T. Zhou, R.-B. Incesu, B. Z. Fu, P. N. P. Gnanapragasam, T. Y. Oliveira, M. S. Seaman, P. D. Kwong, P. J. Bjorkman, M. C. Nussenzweig, Somatic mutations of the immunoglobulin framework are generally required for broad and potent HIV-1 neutralization, Cell 153, 126-138 (2013).
8. D. R. Burton, R. Ahmed, D. H. Barouch, S. T. Butera, S. Crotty, A. Godzik, D. E. Kaufmann, M. J. McElrath, M. C. Nussenzweig, B. Pulendran, C. N. Scanlan, W. R. Schief, G. Silvestri, H. Streeck, B. D. Walker, L. M. Walker, A. B. Ward, I. A. Wilson, R. Wyatt, A Blueprint for HIV Vaccine Discovery, Cell Host and Microbe 12, 396-407 (2012).
9. R. J. Johnston, A. C. Poholek, D. DiToro, I. Yusuf, D. Eto, B. Barnett, A. L. Dent, J. Craft, S. Crotty, Bcl6 and Blimp-1 are reciprocal and antagonistic regulators of T follicular helper cell differentiation, Science 325, 1006-1010 (2009).
10. R. I. Nurieva, Y. Chung, G. J. Martinez, X. O. Yang, S. Tanaka, T. D. Matskevitch, Y. H. Wang, C. Dong, Bcl6 Mediates the Development of T Follicular Helper Cells, Science 325, 1001-1005 (2009).
11. D. Yu, S. Rao, L. M. Tsai, S. K. Lee, Y. He, E. L. Sutcliffe, M. Srivastava, M. Linterman, L. Zheng, N. Simpson, J. I. Ellyard, I. A. Parish, C. S. Ma, Q.-J. J. Li, C. R. Parish, C. R. Mackay, C. G. Vinuesa, The transcriptional repressor Bcl-6 directs T follicular helper cell lineage commitment, Immunity 31, 457-468 (2009).
12. A. D. Gitlin, Z. Shulman, M. C. Nussenzweig, Clonal selection in the germinal centre by regulated proliferation and hypermutation, Nature 509, 637-640 (2014).
13. S. Crotty, Follicular helper CD4 T cells (TFH), Annu. Rev. Immunol. 29, 621-663 (2011).
14. S. Crotty, T follicular helper cell differentiation, function, and roles in disease, Immunity 41, 529-542 (2014).
15. K. M. Ansel, V. N. Ngo, P. L. Hyman, S. A. Luther, R. Forster, J. D. Sedgwick, J. L. Browning, M. Lipp, J. G. Cyster, A chemokine-driven positive feedback loop organizes lymphoid follicles, Nature 406, 309-314 (2000).
16. N. M. Haynes, C. D. C. Allen, R. Lesley, K. M. Ansel, N. Killeen, J. G. Cyster, Role of CXCR5 and CCR7 in follicular Th cell positioning and appearance of a programmed cell death gene-1high germinal center-associated subpopulation, J. Immunol. 179, 5099-5108 (2007).
17. C. D. C. Allen, T. Okada, J. G. Cyster, Germinal-Center Organization and Cellular Dynamics, Immunity 27, 190-202 (2007).
18. X. Wang, B. Cho, K. Suzuki, Y. Xu, J. A. Green, J. An, J. G. Cyster, Follicular dendritic cells help establish follicle identity and promote B cell retention in germinal centers, Journal of Experimental Medicine 208, 2497-2510 (2011).
19. M. A. Kroenke, D. Eto, M. Locci, M. Cho, T. Davidson, E. K. Haddad, S. Crotty, Bcl6 and Maf cooperate to instruct human follicular helper CD4 T cell differentiation, The Journal of Immunology 188, 3734-3744 (2012).
20. A.-U. Rasheed, H.-P. Rahn, F. Sallusto, M. Lipp, G. Müller, Follicular B helper T cell activity is confined to CXCR5(hi)ICOS(hi) CD4 T cells and is independent of CD57 expression, Eur. J. Immunol. 36, 1892-1903 (2006).
21. J. Sáez de Guinoa, L. Barrio, M. Mellado, Y. R. Carrasco, CXCL13/CXCR5 signaling enhances BCR-triggered B-cell activation by shaping cell dynamics, Blood 118, 1560-1569 (2011).
22. X. Wang, H. Yuling, J. Yanping, T. Xinti, Y. Yaofang, Y. Feng, X. Ruijin, W. Li, C. Lang, L. Jingyi, T. Zhiqing, O. Jingping, X. Bing, Q. Li, A. E. Chang, Z. Sun, J. Youxin, T. Jinquan, CCL19 and CXCL13 synergistically regulate interaction between B cell acute lymphocytic leukemia CD23+CD5+ B Cells and CD8+ T cells, J. Immunol. 179, 2880-2888 (2007).
23. D. P. Widney, E. C. Breen, W. J. Boscardin, S. G. Kitchen, J. M. Alcantar, J. B. Smith, J. A. Zack, R. Detels, O. Martínez-Maza, Serum Levels of the Homeostatic B Cell Chemokine, CXCL13, Are Elevated During HIV Infection, Journal of Interferon & Cytokine Research 25, 702-706 (2005).
24. D. Sansonno, F. A. Tucci, L. Troiani, G. Lauletta, M. Montrone, V. Conteduca, L. Sansonno, F. Dammacco, Increased serum levels of the chemokine CXCL13 and up-regulation of its gene expression are distinctive features of HCV-related cryoglobulinemia and correlate with active cutaneous vasculitis, Blood 112, 1620-1627 (2008).
25. H. T. Lee, Y. M. Shiao, T. H. Wu, W. S. Chen, Y. H. Hsu, S. F. Tsai, C. Y. Tsai, Serum BLC/CXCL13 Concentrations and Renal Expression of CXCL13/CXCR5 in Patients with Systemic Lupus Erythematosus and Lupus Nephritis, The Journal of Rheumatology 37, 45-52 (2009).
26. C. K. Wong, P. T. Y. Wong, L. S. Tam, E. K. Li, D. P. Chen, C. W. K. Lam, Elevated Production of B Cell Chemokine CXCL13 is Correlated with Systemic Lupus Erythematosus Disease Activity, J Clin Immunol 30, 45-52 (2009).

27. J. Panse, K. Friedrichs, A. Marx, Y. Hildebrandt, T. Luetkens, K. Barrels, C. Horn, T. Stahl, Y. Cao, K. Milde-Langosch, A. Niendorf, N. Kröger, S. Wenzel, R. Leuwer, C. Bokemeyer, S. Hegewisch-Becker, D. Atanackovic, Chemokine CXCL13 is overexpressed in the tumour tissue and in the peripheral blood of breast cancer patients, Br. J. Cancer 99, 930-938 (2008).
28. Y. Guan, Immunologic basis for long HCDR3s in broadly neutralizing antibodies against HIV-1, 1-8 (2014).
29. M. Locci, C. Havenar-Daughton, E. Landais, J. Wu, M. A. Kroenke, C. L. Arlehamn, L. F. Su, R. Cubas, M. M. Davis, A. Sette, E. K. Haddad, P. Poignard, S. Crotty, I. A. V. I. P. C. P. Investigators89, Human Circulating PD-1+CXCR3−CXCR5+ Memory Tfh Cells Are Highly Functional and Correlate with Broadly Neutralizing HIV Antibody Responses, Immunity, 1-12 (2013).
30. A. J. Hessell, N. L. Haigwood, Neutralizing Antibodies and Control of HIV: Moves and Countermoves, Curr HIV/AIDS Rep 9, 64-72 (2011).
31. K. Cohen, M. Altfeld, G. Alter, L. Stamatatos, Early preservation of CXCR5+ PD-1+ helper T cells and B cell activation predict the breadth of neutralizing antibody responses in chronic HIV-1 infection, J. Virol., JVI.02186-14 (2014).
32. K. W. Cohen, A.-S. Dugast, G. Alter, M. J. McElrath, L. Stamatatos, HIV-1 Single-Stranded RNA Induces CXCL13 Secretion in Human Monocytes via TLR7 Activation and Plasmacytoid Dendritic Cell-Derived Type I IFN, J. Immunol., 1400952 (2015).
33. H. Yu, A. Shahsafaei, D. M. Dorfman, Germinal-center T-helper-cell markers PD-1 and CXCL13 are both expressed by neoplastic cells in angioimmunoblastic T-cell lymphoma, Am. J. Clin. Pathol. 131, 33-41 (2009).
34. F. O. Martinez, L. Helming, S. Gordon, Alternative Activation of Macrophages: An Immunologic Functional Perspective, Annu. Rev. Immunol. 27, 451-483 (2009).
35. S. Ishikawa, T. Sato, M. Abe, S. Nagai, N. Onai, H. Yoneyama, Y. Zhang, T. Suzuki, S. Hashimoto, T. Shirai, M. Lipp, K. Matsushima, Aberrant high expression of B lymphocyte chemokine (BLC/CXCL13) by C11b+CD11c+ dendritic cells in murine lupus and preferential chemotaxis of B1 cells towards BLC, J Exp Med 193, 1393-1402 (2001).
36. J. Jardine, J.-P. Julien, S. Menis, T. Ota, O. Kalyuzhniy, A. McGuire, D. Sok, P.-S. Huang, S. MacPherson, M. Jones, T. Nieusma, J. Mathison, D. Baker, A. B. Ward, D. R. Burton, L. Stamatatos, D. Nemazee, I. A. Wilson, W. R. Schief, Rational HIV immunogen design to target specific germline B cell receptors, Science 340, 711-716 (2013).
37. J. D. Ahlers, All Eyes on the Next Generation of HIV Vaccines: Strategies for Inducing a Broadly Neutralizing Antibody Response, Discovery Medicine 17, 187-199 (2014).
38. R. W. Sanders, M. J. van Gils, R. Derking, D. Sok, T. J. Ketas, J. A. Burger, G. Ozorowski, A. Cupo, C. Simonich, L. Goo, H. Arendt, H. J. Kim, J. H. Lee, P. Pugach, M. Williams, G. Debnath, B. Moldt, M. J. van Breemen, G. Isik, M. Medina-Ramírez, J. W. Back, W. C. Koff, J.-P. Julien, E. G. Rakasz, M. S. Seaman, M. Guttman, K. K. Lee, P.-J. Klasse, C. Labranche, W. R. Schief, I. A. Wilson, J. Overbaugh, D. R. Burton, A. B. Ward, D. C. Montefiori, H. Dean, J. P. Moore, HIV-1 neutralizing antibodies induced by native-like envelope trimers, Science, aac4223 (2015).
39. C. G. Vinuesa, M. C. Cook, C. Angelucci, V. Athanasopoulos, L. Rui, K. M. Hill, D. Yu, H. Domaschenz, B. Whittle, T. Lambe, I. S. Roberts, R. R. Copley, J. I. Bell, R. J. Cornall, C. C. Goodnow, A RING-type ubiquitin ligase family member required to repress follicular helper T cells and autoimmunity, Nature 435, 452-458 (2005).
40. J. J. Hong, P. K. Amancha, K. A. Rogers, C. L. Courtney, C. Havenar-Daughton, S. Crotty, A. A. Ansari, F. Villinger, Early lymphoid responses and germinal center formation correlate with lower viral load set points and better prognosis of simian immunodeficiency virus infection, The Journal of Immunology 193, 797-806 (2014).
41. H. Xu, X. Wang, A. A. Lackner, R. S. Veazey, PD-1(HIGH) Follicular CD4 T Helper Cell Subsets Residing in Lymph Node Germinal Centers Correlate with B Cell Maturation and IgG Production in Rhesus Macaques, Front Immunol 5, 85 (2014).
42. D. Furman, V. Jojic, B. Kidd, S. Shen-Orr, J. Price, J. Jarrell, T. Tse, H. Huang, P. Lund, H. T. Maecker, P. J. Utz, C. L. Dekker, D. Koller, M. M. Davis, Apoptosis and other immune biomarkers predict influenza vaccine responsiveness, Mol. Syst. Biol. 9, 659-659 (2013).
43. S. C. De Rosa, E. P. Thomas, J. Bui, Y. Huang, A. deCamp, C. Morgan, S. A. Kalams, G. D. Tomaras, R. Akondy, R. Ahmed, C.-Y. Lau, B. S. Graham, G. J. Nabel, M. J. McElrath, National Institute of Allergy and Infectious Diseases HIV Vaccine Trials Network, HIV-DNA priming alters T cell responses to HIV-adenovirus vaccine even when responses to DNA are undetectable, The Journal of Immunology 187, 3391-3401 (2011).
44. N. Simpson, P. A. Gatenby, A. Wilson, S. Malik, D. A. Fulcher, S. G. Tangye, H. Manku, T. J. Vyse, G. Roncador, G. A. Huttley, C. C. Goodnow, C. G. Vinuesa, M. C. Cook, Expansion of circulating T cells resembling follicular helper T cells is a fixed phenotype that identifies a subset of severe systemic lupus erythematosus, Arthritis Rheum 62, 234-244 (2010).
45. S. E. Bentebibel, S. Lopez, G. Obermoser, N. Schmitt, C. Mueller, C. Harrod, E. Flano, A. Mejias, R. A. Albrecht, D. Blankenship, H. Xu, V. Pascual, J. Banchereau, A. Garcia-Sastre, A. K. Palucka, O. Ramilo, H. Ueno, Induction of ICOS+CXCR3+CXCR5+ TH Cells Correlates with Antibody Responses to Influenza Vaccination, Science Translational Medicine 5, 176ra32-176ra32 (2013).
46. L. J. Thompson, G. A. Kolumam, S. Thomas, K. Murali-Krishna, Innate inflammatory signals induced by various pathogens differentially dictate the IFN-I dependence of CD8 T cells for clonal expansion and memory formation, J. Immunol. 177, 1746-1754 (2006).
47. M. J. Johnson, C. Petrovas, T. Yamamoto, R. W. B. Lindsay, K. Loré, J. G. D. Gall, E. Gostick, F. Lefebvre, M. J. Cameron, D. A. Price, E. Haddad, R.-P. Sékaly, R. A. Seder, R. A. Koup, Type I IFN induced by adenovirus serotypes 28 and 35 has multiple effects on T cell immunogenicity, The Journal of Immunology 188, 6109-6118 (2012).
48. D. D. Richman, T. Wrin, S. J. Little, C. J. Petropoulos, Rapid evolution of the neutralizing antibody response to HIV type 1 infection, Proc. Natl. Acad. Sci. U.S.A. 100, 4144-4149 (2003).
49. H.-X. Liao, R. Lynch, T. Zhou, F. Gao, S. M. Alam, S. D. Boyd, A. Z. Fire, K. M. Roskin, C. A. Schramm, Z. Zhang, J. Zhu, L. Shapiro, NISC Comparative Sequencing Program, J. C. Mullikin, S. Gnanakaran, P. Hraber, K. Wiehe, G. Kelsoe, G. Yang, S.-M. Xia, D. C. Montefiori, R. Parks, K. E. Lloyd, R. M. Scearce, K. A. Soderberg, M. Cohen, G. Kamanga, M. K. Louder, L. M. Tran, Y. Chen, F. Cai, S. Chen, S. Moquin, X. Du, M. G. Joyce, S. Srivatsan, B. Zhang, A. Zheng, G. M. Shaw, B. H. Hahn, T. B. Kepler, B. T. M. Korber, P. D. Kwong, J. R. Mascola, B. F. Haynes, Co-evolution of a broadly neutralizing HIV-1 antibody and founder virus, Nature 496, 469-476 (2013).
50. K. L. Boswell, R. Paris, E. Boritz, D. Ambrozak, T. Yamamoto, S. Darko, K. Wloka, A. Wheatley, S. Narpala, A. McDermott, M. Roederer, R. Haubrich, M. Connors, J. Ake, D. C. Douek, J. Kim, C. Petrovas, R. A. Koup, Loss of circulating CD4 T cells with B cell helper function during chronic HIV infection, PLoS Pathog 10, e1003853 (2014).
51. D. K. Finch, R. Ettinger, J. L. Karnell, R. Herbst, M. A. Sleeman, Effects of CXCL13 inhibition on lymphoid follicles in models of autoimmune disease, Eur J Clin Invest 43, 501-509 (2013).
52. J. M. Kramer, E. Klimatcheva, T. L. Rothstein, CXCL13 is elevated in Sjögren's syndrome in mice and humans and is implicated in disease pathogenesis, Journal of Leukocyte Biology 94, 1079-1089 (2013).
53. Y.-M. Shiao, C.-C. Lee, Y.-H. Hsu, S.-F. Huang, C.-Y. Lin, L.-H. Li, C. S.-J. Fann, C.-Y. Tsai, S.-F. Tsai, H.-C. Chiu, Ectopic and high CXCL13 chemokine expression in myasthenia gravis with thymic lymphoid hyperplasia, J. Neuroimmunol. 221, 101-106 (2010).
54. I. Rioja, F. J. Hughes, C. H. Sharp, L. C. Warnock, D. S. Montgomery, M. Akil, A. G. Wilson, M. H. Binks, M. C. Dickson, Potential novel biomarkers of disease activity in rheumatoid arthritis patients: CXCL13, CCL23, transforming growth factor α, tumor necrosis factor receptor superfamily member 9, and macrophage colony-stimulating factor, Arthritis Rheum 58, 2257-2267 (2008).
55. E. Klimatcheva, T. Pandina, C. Reilly, S. Torno, H. Bussler, M. Scrivens, A. Jonason, C. Mallow, M. Doherty, M. Paris, E. S. Smith, M. Zauderer, CXCL13 antibody for the treatment of autoimmune disorders, BMC Immunol. 16, 6 (2015).
56. K. Moreth, R. Brodbeck, A. Babelova, N. Gretz, T. Spieker, J. Zeng-Brouwers, J. Pfeilschifter, M. F. Young, R. M. Schaefer, L. Schaefer, The proteoglycan biglycan regulates expression of the B cell chemoattractant CXCL13 and aggravates murine lupus nephritis, J. Clin. Invest. 120, 4251-4272 (2010).
57. C. Pitzalis, G. W. Jones, M. Bombardieri, S. A. Jones, Ectopic lymphoid-like structures in infection, cancer and autoimmunity, Nature Reviews Immunology 14, 447-462 (2014).
58. M. D. Simek, W. Rida, F. H. Priddy, P. Pung, E. Carrow, D. S. Laufer, J. K. Lehrman, M. Boaz, T. Tarragona-Fiol, G. Miiro, J. Birungi, A. Pozniak, D. A. McPhee, O. Manigart, E. Karita, A. Inwoley, W. Jaoko, J. Dehovitz, L.-G. Bekker, P. Pitisuttithum, R. Paris, L. M. Walker, P. Poignard, T. Wrin, P. E. Fast, D. R. Burton, W. C. Koff, Human immunodeficiency virus type 1 elite neutralizers: individuals with broad and potent neutralizing activity identified by using a high-throughput neutralization assay together with an analytical selection algorithm, J. Virol. 83, 7337-7348 (2009).
59. M. Lindqvist, J. van Lunzen, D. Z. Soghoian, B. D. Kuhl, S. Ranasinghe, G. Kranias, M. D. Flanders, S. Cutler, N. Yudanin, M. I. Muller, I. Davis, D. Farber, P. Hartjen, F. Haag, G. Alter, J. Schulze zur Wiesch, H. Streeck, Expansion of HIV-specific T follicular helper cells in chronic HIV infection, J. Clin. Invest. 122, 3271-3280 (2012).
60. D. Furman, B. P. Hejblum, N. Simon, V. Jojic, C. L. Dekker, R. Thiébaut, R. J. Tibshirani, M. M. Davis, Systems analysis of sex differences reveals an immunosuppressive role for testosterone in the response to influenza vaccination, Proceedings of the National Academy of Sciences 111, 869-874 (2014).
61. S. Edupuganti, R. B. Eidex, H. Keyserling, R. S. Akondy, R. Lanciotti, W. Orenstein, C. del Rio, Y. Pan, T. Querec, H. Lipman, A. Barrett, R. Ahmed, D. Teuwen, M. Cetron, M. J. Mulligan, YF-Ig Study Team, A randomized, double-blind, controlled trial of the 17D yellow fever virus vaccine given in combination with immune globulin or placebo: comparative viremia and immunogenicity, Am. J. Trop. Med. Hyg. 88, 172-177 (2013).
62. J. P. Nance, S. Crotty, Cutting Edge: T Follicular Helper Cell Differentiation Is Defective in the Absence of Bcl6 BTB Repressor Domain Function, J. Immunol., 1-10 (2015).
63. S. P. Kasturi, I. Skountzou, R. A. Albrecht, D. Koutsonanos, T. Hua, H. I. Nakaya, R. Ravindran, S. Stewart, M. Alam, M. Kwissa, F. Villinger, N. Murthy, J. Steel, J. Jacob, R. J. Hogan, A. García-Sastre, R. Compans, B. Pulendran, Programming the magnitude and persistence of antibody responses with innate immunity, Nature 470, 543-547 (2011).
64. C. M. Lucero, B. Fallert Junecko, C. R. Klamar, L. A. Sciullo, S. J. Berendam, A. R. Cillo, S. Qin, Y. Sui, S. Sanghavi, M. A. Murphey-Corb, T. A. Reinhart, Macaque Paneth Cells Express Lymphoid Chemokine CXCL13 and Other Antimicrobial Peptides Not Previously Described as Expressed in Intestinal Crypts, Clinical and Vaccine Immunology 20, 1320-1328 (2013).
65. Y. S. Choi, J. A. Yang, I. Yusuf, R. J. Johnston, J. Greenbaum, B. Peters, S. Crotty, Bcl6 expressing follicular helper CD4 T cells are fate committed early and have the capacity to form memory, The Journal of Immunology 190, 4014-4026 (2013).

### Example 3—Certain Embodiments

A1. A method of detecting increased germinal center activity in a subject, comprising:
   a) providing a subject;
   b) measuring the amount of CXCL13 in the subject;
   c) administering an antigen or vaccine to the subject;
   d) measuring the amount of CXCL13 in the subject administered the antigen or vaccine thereby providing a post-treatment level of CXCL13, and
   e) comparing the amount of CXCL13 prior to and after the antigen or vaccine is administered, wherein increased CXCL13 after administration to the subject detects increased germinal center activity.
A2. A method of detecting increased Tfh cell activity in a subject, comprising:
   a) providing a subject;
   b) measuring the amount of CXCL13 in the subject;
   c) administering a antigen or vaccine to the subject;
   d) measuring the amount of CXCL13 in the subject administered the antigen or vaccine thereby providing a post-treatment level of CXCL13, and
   e) comparing the amount of CXCL13 prior to and after the antigen or vaccine was administered, wherein increased CXCL13 after administration to the subject detects increased Tfh cell activity.
A3. A method of detecting responsiveness of a subject to an antigen or a vaccine comprising:
   a) providing a subject;
   b) measuring the amount of CXCL13 in the subject thereby providing a pre-vaccine level of CXCL13;
   c) administering an antigen or vaccine to the subject;
   d) measuring the amount of CXCL13 in the subject thereby providing a post-antigen or vaccine level of CXCL13, and
   e) detecting the responsiveness of the subject to the antigen or vaccine according to the amount of the pre-antigen or vaccine level of CXCL13 to the post-antigen or vaccine amount of CXCL13.
A4. A method of conducting a vaccine trial comprising:
   a) providing two or more study groups comprising subjects, said first study group designated treated and said second study group designated untreated;
   b) measuring the amount of CXCL13 in the subjects thereby providing a pre-vaccine level of CXCL13;
   c) administering a test vaccine to subject(s) of the first study group and a placebo to subject(s) of the second study group;
   d) measuring the amount of CXCL13 in the subjects thereby providing a post-treatment level of CXCL13, and
   e) determining the efficacy of the vaccine according to the ratio of the pre-vaccine level of CXCL13 to the post-treatment level of CXCL13 in the subjects of the two or more study groups.
A5. The method of any of embodiments A1 to A4, wherein a ratio of the pre-antigen or vaccine level of CXCL13 to the post-antigen or vaccine level of CXCL13 is determined.
A6. A method of treating a subject with an autoimmune disorder comprising:
   a) providing a subject having an autoimmune disorder wherein the disorder is characterized by the presence of autoantibodies;
   b) measuring the amount of plasma CXCL13 in the subject thereby providing a pre-treatment level of CXCL13;
   c) administering a dose of a drug to the subject;
   d) measuring the amount of CXCL13 in the subject thereby providing a post-treatment level of CXCL13,
   wherein if the post-treatment level of CXCL13 is less than the pre-treatment level of CXCL13, continue administering the drug, or
   wherein if the post-treatment level of CXCL13 is the same or larger than the pre-treatment level of CXCL13, discontinue administering the drug, or decrease the dose of the drug administered.
A7. The method of any one of embodiments A1 to A6, wherein the CXCL13 is measured about 1 week or more after administering the antigen, vaccine and/or placebo.
A8. The method of any of embodiments A1 to A6, wherein plasma or blood CXCL13 levels are measured.
A9. The method of any one of embodiments A1 to A6, wherein the CXCL13 is measured using a binding agent.
A10. The method of embodiment A9, wherein the binding agent comprises an antibody or a fragment of an antibody that binds to CXCL13.
A11. The method of any one of embodiments A1 to A6, wherein the antigen or vaccine comprises a pathogen.
A12. The method of any of one of embodiments A1 to A6, wherein the antigen or vaccine comprises a live, dead or attenuated virus, bacteria, fungus or parasite.
A13. The method of any of one of embodiments A1 to A6, wherein the antigen or vaccine comprises a viral, bacterial, fungal or parasite extract or protein.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It will be understood by those of skill in the art that numerous and various modifications can be made without departing from the spirit of the technology. Therefore, it should be clearly understood that the forms of the technology are illustrative only and are not intended to limit the scope of the technology.

What is claimed:

1. A method of detecting an immune response in a subject to an antigen or vaccine, comprising:
a) determining a first amount of CXCL13 in a first sample obtained from a subject;
b) administering an antigen or vaccine to the subject;
c) determining a second amount of CXCL13 in a second sample obtained from the subject after the administering in (b); and
d) comparing the first and the second amount of CXCL13, wherein a presence or absence of an immune response to the antigen or vaccine is determined according to the comparison.

2. The method of claim 1, wherein the comparison comprises determining the presence or absence of an increase in the second amount of CXCL13 compared to the first amount of CXCL13.

3. The method of claim 2, wherein the presence of an increase in the second amount of CXCL13 indicates the presence of an immune response to the antigen or vaccine.

4. The method of claim 3, wherein the presence of the immune response to the antigen or vaccine comprises an increase in germinal center (GC) activity.

5. The method of claim 4, wherein the increase in germinal center (GC) activity comprises at least a 10% increase in the amount of germinal centers in one or more lymph node tissues in the subject.

6. The method of claim 4, wherein the increase in germinal center (GC) activity comprises at least a 10% increase in the amount of Tfh cells in one or more lymph node tissue in the subject.

7. The method of claim 6, wherein the Tfh cells produce CXCL13.

8. The method of claim 4, wherein the increase in germinal center (GC) activity comprises at least a 10% increase in the amount of circulating activated T-cells in the subject, wherein the activated T-cells are ICOS⁺PD1⁺CXCR5⁺CD4⁺.

9. The method of any one of claims 1 to 8, wherein the comparing in (d) comprises determining a ratio of the first amount of CXCL13 to the second amount of CXCL13.

10. The method of any one of claims 1 to 9, wherein the second sample is obtained at least 1 week after administering the antigen or vaccine.

11. The method of any one of claims 1 to 10, wherein the first and the second sample comprise or consist essentially of blood, plasma or serum obtained from the patient.

12. The method of any one of claims 1 to 11, wherein the determining of (b) and (c) comprises use of a binding agent that specifically binds to CXCL13.

13. The method of claim 12, wherein the binding agent comprises an antibody or a fragment of an antibody that binds to specifically to CXCL13.

14. The method of any of claims 1 to 13, wherein the antigen or vaccine comprises a pathogen, or an immunogenic portion thereof.

15. The method of claim 14, wherein the pathogen comprises a live, dead or attenuated virus, bacteria, fungus or parasite.

16. The method of claim 14, wherein the immunogenic portion of the pathogen comprises a viral, bacterial, fungal or parasite extract or protein.

17. The method of claim 3, wherein the presence of an immune response indicates the presence of circulating antibodies in the subject that bind specifically to the antigen, or to an antigen component of the vaccine.

18. The method of claim 17, wherein the amount of circulating antibodies in the subject after the administering of (b) is substantially larger than an amount of circulating antibodies in the subject prior to the administering of (b).

19. The method of any one of claims 2 to 18, wherein the presence of an increase in the second amount of CXCL13 compared to the first amount of CXCL13 is at least a 10% increase in the second amount of CXCL13 compared to the first amount of CXCL13.

20. The method of claim 19, wherein the at least 10% increase is at least a 30% increase.

21. The method of claim 19, wherein the at least 10% increase is at least a 2-fold increase.

22. The method of claim 2, wherein the absence of an increase in the second amount of CXCL13 indicates the absence of an immune response to the antigen or vaccine.

23. The method of claim 22, wherein the absence of an immune response indicates the subject is not responsive to the antigen or vaccine.

24. The method of claim 22, wherein the absence of an immune response indicates the subject is immuno-suppressed or immuno-incompetent.

25. The method of claim 3, wherein the presence of an immune response indicates the vaccine is effective.

26. A method of treating a subject with an autoimmune disorder comprising:
a) providing a subject having an autoimmune disorder wherein the disorder is characterized by the presence of autoantibodies;
b) determining a first amount of CXCL13 in a sample obtained from the subject;
c) administering a dose of a drug to the subject, wherein the drug is configured to treat the autoimmune disorder;
d) determining a second amount of CXCL13 in a second sample obtained from the subject after the administering of (c); and
e) comparing the first amount to the second amount thereby providing a comparison.

27. The method of claim 26, wherein if the second amount of CXCL13 is less than the first amount of CXCL13, continue administering the drug.

28. The method of claim 26, wherein if the second amount of CXCL13 is the same or larger than the first amount of CXCL13, discontinue administering the drug, or increase the dose of the drug administered.

29. A method of diagnosing a subject with an autoimmune disorder comprising:
a) providing a first sample comprising a known amount of CXCL13;
b) determining an amount of CXCL13 in a second sample obtained from a subject having or suspected of having an autoimmune disorder;
c) comparing the amount of CXCL13 in the first sample to the second sample, thereby providing a comparison; and
d) determining the presence or absence of an autoimmune disorder in the subject according to the comparison.

30. The method of claim 29, wherein the presence of an autoimmune disorder is determined and the amount of CXCL13 in the second sample is at least 30% greater than the amount of CXCL13 in the first sample.

31. The method of claim 29, wherein the absence of an autoimmune disorder is determined and the amount of CXCL13 in the second sample is not significantly different than the amount of CXCL13 in the first sample.

32. The method of claim 29, wherein the autoimmune disorder is characterized by the presence of autoantibodies.

33. The method of claim 29, wherein the autoimmune disorder is systemic lupus erythematosus or rheumatoid arthritis.

34. The method of any one of claims 1 to 33, wherein the sample comprises or consists essentially of blood, plasma or serum obtained from the subject, and wherein the subject is a human subject.

35. A method of detecting increased germinal center activity in a subject, comprising:
a) providing a subject;
b) measuring an amount of CXCL13 in a sample obtained from the subject;
c) measuring an amount of CXCL13 in a control sample;
d) comparing the amount of CXCL13 in the subject to the amount to CXCL13 in the control sample, wherein increased germinal center activity in the subject is determined according to the comparison.

36. The method of claim 35, wherein the comparison comprises determining the presence or absence of an increase in the amount CXCL13 in the sample obtained from the subject compared to the amount of CXCL13 in the control sample.

37. The method of claim 35, wherein the presence of an increase in the amount of CXCL13 in the sample obtained from the subject indicates increased germinal center activity in the subject.

38. The method of claim 35, wherein the subject has, is suspected of having, or is at risk of having an autoimmune disorder.

39. A method of detecting increased Tfh cell activity in a subject, comprising:
a) providing a subject;
b) measuring an amount of CXCL13 in a sample obtained from the subject;
c) measuring an amount of CXCL13 in a control sample;
d) comparing the amount of CXCL13 in the subject to the amount to CXCL13 in the control sample, wherein increased Tfh activity in the subject is determined according to the comparison.

40. The method of claim 39, wherein the comparison comprises determining the presence or absence of an increase in the amount of CXCL13 in the sample obtained from the subject compared to the amount of CXCL13 in the control sample.

41. The method of claim 40, wherein the presence of an increase of CXCL13 in the sample obtained from the subject indicates increased Tfh activity in the subject.

42. The method of claim 39, wherein the subject has, is suspected of having or is at risk of having an autoimmune disorder.
